SEQUENCE LISTING (2 sequences)

SEQ ID NO: 1 - protein, 418 aa, homo sapiens
  SIGNAL (1)..(19): protein secretion leader sequence
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 2 - protein, 44 aa, artificial
  44-amino acid region of Pigment epithelium-derived factor (PEDF)
VLLSPLSVATALSALSLGAEQRTESIIHRALYYDLISSPDIHGT

(19) United States
(12) Patent Application Publication
(10) Pub. No.: US 2013/0046283 A1
(43) Pub. Date: Feb. 21, 2013

(54) METHODS AND INTRAVASCULAR TREATMENT DEVICES FOR TREATMENT OF ATHEROSCLEROSIS

(71) Applicants: Ayala Hezi-Yamit, Santa Rosa, CA (US); Susan Edwards, Santa Rosa, CA (US); Carol Sullivan, Petaluma, CA (US); Oluwashola Shakirat Sulaimon, Santa Rosa, CA (US); Lori Garcia, Santa Rosa, CA (US); Stefan Tunev, Santa Rosa, CA (US)

(72) Inventors: Ayala Hezi-Yamit, Santa Rosa, CA (US); Susan Edwards, Santa Rosa, CA (US); Carol Sullivan, Petaluma, CA (US); Oluwashola Shakirat Sulaimon, Santa Rosa, CA (US); Lori Garcia, Santa Rosa, CA (US); Stefan Tunev, Santa Rosa, CA (US)

(73) Assignee: Medtronic Vascular, Inc., Santa Rosa, CA (US)

(21) Appl. No.: 13/464,907

(22) Filed: May 4, 2012

Related U.S. Application Data
(60) Provisional application No. 61/482,770, filed on May 5, 2011.

(51) Int. Cl. A61K 38/17; A61M 25/10; A61K 9/00; A61P 9/10; A61K 31/4375

(52) U.S. Cl. 604/509; 514/1.9; 514/287; 424/400; 604/96.01

(57) ABSTRACT

Methods and intravascular treatment devices for treating atherosclerosis are provided.

### CROSS-REFERENCE TO RELATED APPLICATIONS

This application claims the benefit and priority of U.S. Provisional Application No. 61/482,770 filed May 5, 2011, entitled “METHODS AND INTRAVASCULAR TREATMENT DEVICES FOR TREATMENT OF ATHEROSCLEROSIS” and is herein incorporated by reference for all purposes.

### BACKGROUND

Cardiovascular diseases (also referred to herein as arterial or vascular diseases), such as peripheral artery (i.e., arterial) disease (PAD), coronary artery (i.e., arterial) disease (CAD), and carotid artery (i.e., arterial) disease, are caused by narrowed or blocked arteries or veins in various regions of the body. They restrict the flow of blood due to, for example, atherosclerosis or inflammatory processes leading to stenosis, an embolism, or thrombus formation, which can result in either acute or chronic ischemia (lack of blood supply). Atherosclerosis is a progressive, dynamic inflammatory disorder characterized by the accumulation of lipids, cells, and extracellular matrix in the vessel walls, i.e., in the inner linings of the walls of the arteries or veins, which limit or obstruct coronary blood flow. Such atherosclerotic lesions (or plaque) are the major cause of ischemic heart disease.

PAD refers to narrowing of peripheral arteries, i.e., those arteries in the outer regions of the arterial system away from the heart and brain, particularly arteries leading to the kidneys, stomach, legs, arms, and feet, due to the build-up of atherosclerotic plaque. CAD typically refers to arteries that directly feed the heart muscle. Carotid artery disease refers arteries that supply blood to the brain.

Percutaneous transluminal coronary angioplasty is a medical procedure whose purpose is to increase blood flow through an artery. Percutaneous transluminal coronary angioplasty is the predominant treatment for coronary vessel stenosis. The increasing use of this procedure is attributable to its relatively high success rate and its minimal invasiveness compared with coronary bypass surgery. Also, the implantation of stents has gained widespread use to maintain increased blood flow. In both cases, however, in many instances re-occlusion due to restenosis occurs. Therapeutic agent (or drug) eluting balloons (DEB) and stents (DES) are known and have been on the market for several years now with excellent clinical success. Therapeutic agent eluting balloons and stents have revolutionized the vascular and cardiologic medicine, aiding in such complications as vulnerable plaque rupture, stenosis, restenosis, ischemic myocardial infarct, and atherosclerosis. However, as with any evolving technology, there is still a need for addressing problems of atherosclerosis.

### SUMMARY

The present disclosure provides methods and intravascular treatment devices for treating atherosclerosis associated with, e.g., cardiovascular diseases. Such atherosclerosis can be in peripheral, coronary, or carotid arteries or veins. In certain embodiments, the methods and devices are particularly suited for treating peripheral arterial disease.

The progress achieved in reducing the rate of restenosis for peripheral arterial disease is not as great as that for coronary arterial disease. That is, in sharp contrast to the remarkable advancement obtained with interventional treatment of CAD, the treatment of PAD has not yielded comparable success. The present disclosure is particularly applicable to treating PAD.

Embodiments according to the present disclosure provide localized application of one or more therapeutic agents useful, e.g., to reduce the severity and the progression of atherosclerosis at a site of build-up of atherosclerotic plaque. Certain embodiments include the administration of one or more therapeutic agents as described herein using local delivery. The agent(s) preferably are localized to (adjacent or within) the site of atherosclerotic build-up of plaque (i.e., lesions) by the placement of an intravascular treatment device that is comprised of, or within which is provided, the therapeutic agent(s).

In certain embodiments, the present disclosure provides a method of treating atherosclerosis (preferably, peripheral arterial disease) in a subject, the method comprising: providing an intravascular treatment device comprising one or more (preferably, two or more) therapeutic agents, wherein the one or more therapeutic agents comprise: a compound that increases the concentration of one or more of the anti-inflammatory/anti-proliferative PEDF protein; a compound that increases the concentration of the anti-proliferative KLF4 protein; a compound that increases the concentration of the anti-proliferative/anti-angiogenic/growth factor binder BTG2 protein; a compound that increases the concentration of the anti-proliferative/angiogenesis inhibitor/growth factor binder Perlecan protein; and combinations thereof; and positioning the intravascular treatment device at a site of build-up of atherosclerotic plaque in a blood vessel, wherein the intravascular treatment device contacts the atherosclerotic site under conditions effective to transfer at least a portion of the one or more therapeutic agents to the subject.

In certain embodiments, the present disclosure provides an intravascular treatment device locatable at an atherosclerotic site in a blood vessel; wherein the device comprises one or more therapeutic agents (and supports the atherosclerotic site upon deployment at least temporarily), wherein the one or more (preferably, two or more) therapeutic agents comprise: a compound that increases the concentration of one or more of the anti-inflammatory/anti-proliferative PEDF protein; a compound that increases the concentration of the anti-proliferative KLF4 protein; a compound that increases the concentration of the anti-proliferative/anti-angiogenic/growth factor binder BTG2 protein; a compound that increases the concentration of the anti-proliferative/angiogenesis inhibitor/growth factor binder Perlecan protein; and combinations thereof.

In certain embodiments, the intravascular treatment device further includes a carrier for the one or more therapeutic agents. In certain embodiments described herein, the therapeutic agent/carrier formulation includes a material to ensure the controlled release of the therapeutic agent(s). In certain embodiments, the intravascular treatment device further includes an excipient.

The term “treating” in the context of “treating atherosclerosis” means improving the condition of, reducing the progression of, or reducing the severity of, vascular occlusions. This includes the inhibition or prevention of the initial (i.e., de novo) development of, or further development of, atherosclerosis, including post-interventional restenosis.

As used herein, “subject” and “patient” are used interchangeably, and include mammals, fish, reptiles and birds. Mammals include, but are not limited to, primates, including humans, dogs, cats, goats, sheep, rabbits, pigs, horses and cows.

As used herein, “biocompatible” shall mean any material that does not cause injury or death to the subject or induce an adverse reaction in a subject when placed in intimate contact with the subject's tissues. Adverse reactions include inflammation, infection, fibrotic tissue formation, cell death, or thrombosis.

As used herein, “controlled release” refers to the release of a therapeutic agent from a intravascular treatment device at a predetermined rate. Controlled release implies that the therapeutic agent does not come off the intravascular treatment device sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present disclosure an initial burst of therapeutic agent may be desirable followed by a more gradual release thereafter, or an initial gradual release followed by a subsequent burst. The release rate may be steady state (commonly referred to as “timed release” or zero order kinetics), that is the therapeutic agent is released in even amounts over a predetermined time (with or without an initial burst phase) or may be a gradient release. A gradient release implies that the concentration of therapeutic agent released from the device surface changes over time.

The term “comprises” and variations thereof do not have a limiting meaning where these terms appear in the description and claims.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “a,” “an,” “the,” “at least one,” and “one or more” are used interchangeably. Thus, for example, a device that comprises “a” polymer can be interpreted to mean that the device includes “one or more” polymers.

As used herein, the term “or” is generally employed in its usual sense including “and/or” unless the content clearly dictates otherwise.

The term “and/or” means one or all of the listed elements or a combination of any one or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range (e.g., 1 to 5 includes 1, 1.5, 2, 2.75, 3, 3.80, 4, 5, etc.) including the endpoints.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 depicts a intravascular treatment device, specifically a vascular stent having the coating made in accordance with the teachings of the present disclosure thereon.

FIG. 2 depicts a vascular stent having a coating made in accordance with the teachings of the present disclosure mounted on a suitable delivery device—a balloon catheter.

FIG. 3 depicts a vascular stent 400 having a coating 504 of the present disclosure mounted on a balloon catheter 601.

FIG. 4 depicts a catheter with an expandable balloon.

FIG. 5 depicts representative diagram of patient superficial femoral arteries and site of lesion harvest. Box insert representative of tissue specimen. (A) De novo and restenotic lesions were procured from individual patients in areas outlined with heavy black line.

FIG. 6 depicts a heat map of differentially expressed genes and representative histological staining of peripheral atherectomy samples. mRNA levels measured by qRT-PCR from either de novo or restenotic lesions calibrated against normal donor vessel. Gene expression patterns summarized for de novo (n=25) and restenotic (n=21) lesions (A) proliferation, (B) Inflammation, and (C) Extracellular Matrix. (D) Representative histology of patient samples and control from peripheral SFA tissue. Alpha smooth muscle, PCNA, CD68, Movat and Ki67 stain in atherectomy and control vessel from the SFA.

FIG. 7 depicts quantitative real time polymerase chain reaction (qRT-PCR) gene expression of cell cycle modulators in SFA control and PAD atherectomy samples.

Relative gene expression levels of: (A) BTG2; (B) KLF4; (C) CDKN1B; (D) PEDF; and (E) CDKN2A were determined for de novo and restenotic samples calibrated against non-disease control. Data represented in a box and whiskers plot. Box area represents from 25th to 75th percentile with the horizontal line at the median 50th percentile. Differences between groups determined using the Mann-Whitney rank sum nonparametric unpaired test.

FIG. 8 depicts qRT-PCR gene expression of de novo and restenotic samples harvested from individual patients with progressive disease. Relative amounts of (A) BTG2, (B) KLF4, (C) PEDF, and (D) CDKN2A. “D” de novo and “R” restenotic lesions within the same individual as compared to non-diseased control.

FIG. 9 depicts relative gene expression by qRT-PCR of inflammatory genes. (A) IL6, CYBB, Osteopontin, IL1B, TNF and LY96; (B) CXCR4, CCL5, Cathepsin S and Cathepsin B; (C) TLR1, TLR2, TLR4 and TLR7; and (D) CD11b, VLA4 and VCAM1 mRNA. P values analyzed by Mann Whitney t-test.

FIG. 10 depicts qRT-PCR inflammatory profile of de novo and restenotic samples harvested from individual patients. Relative amounts of (A) IL6 and (B) VCAM1 mRNA quantified in “D” de novo and “R” restenotic lesions within the same individual as compared to non-diseased control.

FIG. 11 depicts qRT-PCR expression of extracellular matrix related genes. Relative expression levels of: (A) Perlican and Versican; (B) SLRPs—Decorin, Fibromodulin, Biglycan and Lumican; (C) Thrombospondin 1, Thrombospondin 2, Thrombospondin 3 and Thrombospondin 4; and (D) CTGF, Col1A1, Collagen 1A2, Col3A1, Col 5A2 in de novo and restenotic patients as compared to non-disease control. p value determined as per the Mann-Whitney rank sum nonparametric unpaired test. Data represented in box and whiskers plot with the horizontal line representing the median 50th percentile.

FIG. 12 depicts qRT-PCR of extracellular Matrix genes in de novo and restenotic samples derived from individual patients. (A) Thrombospondin 2 (B) Collagen 1A1. Patient subset represents individuals with multiple atherectomy interventions; D=de novo, R=resenotic.

FIG. 13 depicts effects of paclitaxel on cellular morphology, gene and protein expression. (A and B) SFA smooth muscle cells from normal donor tissue were treated with Paclitaxel, Everolimus or Sirolimus. Gene expression levels were determined by qRT-PCR for each drug arm calibrated against unstimulated control for (A) CDKN1A and (C) CTGF. (B) SMCs were exposed to Sirolimus or Paclitaxel, fixed and stained with Actin (red) and Ki67 (green). (D) SFA derived smooth muscle cells were treated with Paclitaxel, Everolimus or Sirolimus then stimulated with Colchicinne (CTGF a) or Angiotensin II (CTGF b). Protein levels were detected by western blot using antibodies against CTGF and beta actin. One representative blot of three replicates shown.

FIG. 14 depicts differential effect of paclitaxel versus the Limus family of drugs on expression of cell cycle, proliferation and ECM target genes in SFA smooth muscle cells. Quiescent SFA smooth muscle cells were treated with of Paclitaxel, Zotarolimus or Sirolimus then stimulated with inflammatory cocktail consisting of FBS, TGF Beta and 11_{—}1 beta. Relative gene expression levels were determined by qRT-PCR for each drug treated arm as calibrated against the unstimulated baseline control for (A) BTG2, (B) KLF4, (C) PEDF, (D) Endothelin-1, (E) Thrombospondin-1, and (F) Thrombospondin-3.

### DETAILED DESCRIPTION OF ILLUSTRATIVE EMBODIMENTS

The present disclosure provides methods and devices for treating atherosclerosis. Such methods and devices support or bolster the atherosclerotic site and supply one or more (in some embodiments, a combination of two or more) therapeutic agents to treat the surrounding atherosclerotic plaque.

Applicants have discovered that the pathogenesis of atherosclerosis (particularly peripheral arterial disease) suggests the following mechanisms play a concurrent role in the formation of atherosclerotic plaque: 1) down regulation in inhibitors of cell cycle regulators (cyclin dependent kinase inhibitors (p21 & p27) and PEDF); 2) up regulation of anti apoptotic molecules (p16 and versican); 3) over expression of the CTGF and thrombospondins; 4) over expression of inflammatory cytokines (IL-6) and proteases; and 5) increased extracellular matrix deposition. In particular, Applicants have discovered that the pathogenesis of atherosclerosis (particularly peripheral arterial disease) suggests the following mechanisms play a concurrent role in the formation of atherosclerotic plaque: 1) down-regulation of the gene that expresses PEDF (Pigment Epithelium-Derived Factor); 2) down-regulation of the KLF4 gene; 3) down-regulation of the BTG2 gene; and 4) down-regulation of the gene that expresses the Perlecan protein. Pharmacologically targeting one or more of these mechanisms offers a convenient alternative to surgical intervention alone.

Thus, the present disclosure is directed to the use of one or more therapeutic agents that target one or more of these mechanisms. Preferably, two or more therapeutic agents are used in combination in a treatment protocol. More preferably, three or more therapeutic agents are used in combination in a treatment protocol. These may be used in admixture, e.g., in a mixture of therapeutic agents in a polymer coating on an intravascular treatment device. Alternatively, they may be used in combination, but not in an admixture. For example, they may be applied to different portions of an intravascular treatment device.

The therapeutic agents for use in the present disclosure include those described herein below. They may be in the form or a salt, a free base, a solvate, a protherapeutic agent, or a physiologically active metabolite. They may be in the form of physiologically active compounds and compositions containing such compounds; and their protherapeutic agents, and pharmaceutically acceptable salts and solvates of such compounds and their protherapeutic agents, as well as novel compounds within the scope of formula of these compounds.

In certain embodiments, the present disclosure provides a method of treating atherosclerosis (preferably, peripheral arterial disease) in a subject, the method comprising: providing an intravascular treatment device comprising one or more (preferably, two or more) therapeutic agents, wherein the one or more therapeutic agents described herein; and positioning the intravascular treatment device at a site of build-up of atherosclerotic plaque in a blood vessel, wherein the intravascular treatment device contacts the atherosclerotic site under conditions effective to transfer at least a portion of the one or more therapeutic agents to the subject.

In certain embodiments, the present disclosure provides an intravascular treatment device locatable at an atherosclerotic site in a blood vessel; wherein the device comprises one or more therapeutic agents (and supports the atherosclerotic site upon deployment at least temporarily), wherein the one or more (preferably, two or more) therapeutic agents are described herein.

Embodiments according to the present disclosure provide localized application of one or more therapeutic agents useful to, e.g., reduce the severity and the progression of atherosclerotic plaque. Certain embodiments include the administration of two or more therapeutic agents as described herein using local delivery. The agents are localized to (e.g., adjacent or within) the atherosclerotic site by the placement of an intravascular treatment device that is comprised of, or within which is provided, the therapeutic agent(s).

The one or more therapeutic agents (typically, two or more, and preferably, three or more therapeutic agents) can be incorporated directly into an intravascular treatment device (e.g., incorporated into a polymer for forming a stent or graft, placed inside a double-walled stent graft), into a carrier associated with an intravascular treatment device (e.g., as a coating on a stent or angioplasty balloon), disposed directly on an intravascular treatment device without a carrier (e.g., a polymeric carrier), or combinations thereof. In certain embodiments, the one or more therapeutic agents can be delivered by the intravascular treatment device over time to the local tissue.

In an embodiment in which a carrier is used, the materials to be used for such a carrier can be synthetic organic polymers, natural organic polymers, inorganics, or combinations of these. The physical form of the therapeutic agent with or without a carrier can be a film, sheet, coating, slab, gel, capsule, microparticle, nanoparticle, or combinations of these.

In embodiments of the invention, one or more low molecular weight excipients or “enhancers” can be intermixed with the one or more therapeutic agents. The one or more therapeutic agents can be mixed with low (less than 10,000 g/mole) to medium (10,000 to 25,000 g/mole) weight average molecular weight excipients that include a fatty acid ester of polyethylene glycol, a polyethylene glycol-polyester block copolymer, a fatty acid mono- or di-ester of glycerol, a fatty acid mono-, di-, or poly-ester of trimethylol ethane or trimethylol propane or pentaerythritol, a sugar, a water-soluble polyol, Also included within the term “excipient” are cyclodextrins, clathrates (cage compounds), sometimes referred to as spacer molecules like urea, crown ethers, deoxycholic acid, and cryptands. Various combinations of these can be used if desired. In certain embodiments, the at least one therapeutic agent is mixed with at least one excipient to form a mixture that is disposed on an intravascular treatment device.

Biological modes of delivery, such as gene therapy, viral delivery, RNAi, anti sense, can be used if desired. These modes of delivery have an advantage of providing selected delivery of genetic material (e.g., DNA or RNA) of interest to the cells in vivo.

### Therapeutic Agents

One or more therapeutic agents that target one or more of the mechanisms identified above by Applicants can be used in the present disclosure. Such therapeutic agents include compounds that increase the concentration (e.g., expression) of one or more of the anti-inflammatory/anti-proliferative PEDF protein; compounds that increase the concentration (e.g., expression) of the anti-proliferative KLF4 protein; compounds that increase the concentration (e.g., expression) of the anti-proliferative/anti-angiogenic/growth factor binder BTG2 protein; and compounds that increase the concentration (e.g., expression) of the anti-proliferative/angiogenesis inhibitor/growth factor binder Perlecan protein. Various combinations of such compounds can be used if desired.

PEDF, or Pigment epithelium-derived factor, is also known as serpin F1 (SERPINF1). It is a multifunctional secreted protein that has anti-proliferative and anti-angiogenic functions. Found in vertebrates, this 50 kDa protein, in humans is encoded by the SERPINF1 gene. The full length amino acid sequence (Accession: BAJ83968.1 GI: 326205164) is as follows (SEQ ID NO:1):

| | |
|---|---|
| 1 | mqalvlllci gallghsscq npasppeegs pdpdstgalv eeedpffkvp vnklaaaysn |
| 61 | fgydlyrvrs stspttnvll splsvatals alslgaeqrt esiihralyy dlisspdihg |
| 121 | tykelldtvt apqknlksas rivfekklri kssfvaplek sygtrprvlt gnprldlqei |
| 181 | nnwvqaqmkg klarstkeip deisilllgv ahfkgqwvtk fdsrktsled fyldeertvr |
| 241 | vpmmsdpkav lrygldsdls ckiaqlpltg smsiifflpl kvtqnltlie esltsefihd |
| 301 | idrelktvqa vltvpklkls yegevtkslq emklqslfds pdfskitgkp ikltqvehra |
| 361 | gfewnedgag ttpspglqpa hltfpldyhl nqpfifvlrd tdtgallfig kildprgp |

The N-terminus contains a leader sequence responsible for protein secretion out of the cell at residues 1-19. A 34-mer fragment of PEDF (residues 24-57) was shown to have anti-angiogenic properties, and a 44-mer (residues 58-101) was shown to have neurotrophic properties. A BLAST search reveals a putative receptor binding site exists between residues 75-124. A nuclear localization sequence (NLS) exists about 150 amino acids into the protein. The additional molecular weight is partly due to a single glycosylation site at residue 285. Near the C-terminus, at residues 365-390 lies the reactive center loop (RCL) which is normally involved in serine protease inhibitor activity; however, in PEDF this region does not retain the inhibitory function. The PEDF structure includes 3 beta sheets and 10 alpha helices. PEDF has an asymmetrical charge distribution across the whole protein. One side of the protein is heavily basic and the other side is heavily acidic, leading to a polar 3-D structure.

A 44-amino acid region of PEDF (shown below and referred to herein as “PDF 44” (SEQ ID NO:2)) has been identified to confer both the anti-vasoppermeability and the anti-angiogenic activities. Additionally, 4 amino acids residues glutamte₁₀₁, isoleucine₁₀₃, leucine₁₁₂ and serine₁₁₅ have been identified for both activities and is believed to be useful as a therapeutic agent for cancer and proliferative retinopathy (Int. Pub. No. WO 2005/041887).

The four important amino acid residues in PEDFₚₑₚ:

(SEQ ID NO: 2)
VLLSPLSVATALSALSLGAEQRTESIIHRALYYDLISSPDIHGT

This protein can be used (directly) as a therapeutic agent. Alternatively, an adenovial vector encoding PEDF (such as that disclosed by K. Mod et al., (2001), Journal of Cellular Physiology, 188: 253-263; Int. Pub. No. WO 2005/105155) can be used as the therapeutic agent. A pharmacological composition comprising a source of PEDF (SEQ ID NO:1) or PEDF 44 AA peptide (SEQ ID NO:2) and a suitable diluent, which includes one or more pharmacologically acceptable carriers (such as physiological compatible buffers that may, if needed contain stabilizers such as polyethelene glycol) can be used in accordance with the present disclosure.

KLF4 (Krueppel-Like Factor 4) is an anti-proliferative protein that in humans is encoded by the KLF4 gene. It inhibits proliferation through activation of p21CIP1/Waf1, and direct suppression of cyclin D1 and cyclin B1 gene expression. Klf4 inhibits proliferation through activation of p21Cip1/Waf1, and direct suppression of cyclin D1 and cyclin B1 gene expression. Both Klf4 & Klf5 proteins act on the Klf4 promoter where Klf4 increases expression and Klf5 decreases expression of Klf4 mRNA. Compounds that increase the expression of KLF4 include LOR-253 (Lorus Therapeutics). LOR-253 (formerly LT-253), which has the following structure

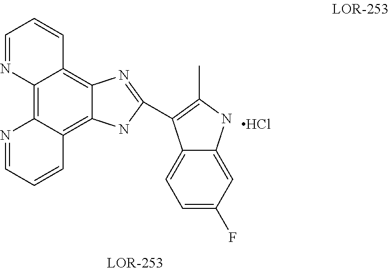 [SMILES: Cc1[nH]c2cc(F)ccc2c1-c1nc2c3cccnc3c3ncccc3c2[nH]1 ; Cl]

is marketed as an anticancer small molecule drug. LOR-253 is a first-in-class inhibitor of the Metal Transcription Factor-1 (MTF-1) with a novel mode of action. This consists of the induction of the tumor suppressor factor Kruppel like factor 4 (KLF4) leading to the down-regulation of cyclin D1, an important regulator of cell cycle progression and cell proliferation, and decreased expression of genes involved in tumor hypoxia (low oxygen content) and angiogenesis.

The protein BTG2, also known as BTG family member 2 or NGF-inducible anti-proliferative protein PC3 or NGF-inducible protein TIS21, is an anti-proliferative protein that in humans is encoded by the BTG2 gene (B-cell translocation gene 2) and in other mammals by the homologous Btg2 gene. The protein encoded by the gene BTG2 (which is the official name assigned to the gene PC3/Tris21/BTG2) is a member of the BTG/Tob family, which has structurally related proteins that appear to have anti-proliferative properties. In particular, the BTG2 protein has been shown to negatively control a cell cycle checkpoint at the G1 to S phase transition in fibroblasts and neuronal cells by direct inhibition of the activity of cyclin D1 promoter.

Perlecan (PLC), also known as basement membrane-specific heparan sulfate proteoglycan core protein (HSPG) or heparan sulfate proteoglycan 2 (HSPG2), is an anti-proliferative protein that in humans is encoded by the HSPG2 gene. Perlecan is a key component of the vascular extracellular matrix, where it interacts with a variety of other matrix components and helps to maintain the endothelial barrier function. Perlecan is a potent inhibitor of smooth muscle cell proliferation and is thus thought to help maintain vascular homeostasis. Perlecan has also been shown to bind many growth factors including BMP-2, CTGF, PDFG, VEGF, several FGF growth factors (e.g., FGF2), and modulate several others. Perlecan is a large multidomain proteoglycan that binds to and cross-links many extracellular matrix (ECM) components and cell-surface molecules. Perlecan is synthesized by both vascular endothelial and smooth muscle cells and deposited in the extracellular matrix.

The dosage of the one or more therapeutic agents described herein will vary depending on the manner in which they are locally delivered. For example, this can depend on the properties of the coating or structure they are incorporated into, including its time-release properties, whether the coating is itself biodegradable, and other properties. Also, the dosage of the one or more therapeutic agents used will vary depending on the potency, pathways of metabolism, extent of absorption, half-life, and mechanisms of elimination of the therapeutic agent itself. In any event, the practitioner is guided by skill and knowledge in the field, and embodiments according to the present disclosure include without limitation dosages that are effective to achieve the described phenomena.

### Intravascular Treatment Devices

Intravascular treatment devices useful in the present disclosure for local delivery of therapeutic agents for the treatment of atherosclerosis as described herein include stents (e.g., vascular stents, coronary artery stents, peripheral vascular stents), stent grafts, angioplasty balloons (i.e., dilatation balloons), and the like. Various intravascular treatment devices can be modified using the one or more therapeutic agents described herein using the teachings of the present disclosure.

Various methods of incorporating the one or more therapeutic agents into an intravascular treatment device can be used. For example, the one or more therapeutic agents can be incorporated directly into an intravascular treatment device (e.g., incorporated into a polymer for forming a stent or stent graft), or into a carrier (e.g., a polymeric material) associated with such intravascular treatment device (e.g., as a coating on a stent or angioplasty balloon), or disposed directly on an intravascular treatment device without a carrier, or combinations thereof.

In certain embodiments, the one or more therapeutic agents are delivered by the intravascular treatment device over time to the local tissue. The materials to be used for such a carrier can be synthetic organic polymers, natural organic polymers, inorganics, or combinations of these. The physical form of the therapeutic agent/carrier formulation can be a film, sheet, coating, slab, gel, capsule, microparticle, nanoparticle, or combinations of these.

In one preferred embodiment of the present disclosure, the intravascular treatment device is a vascular stent. Therapeutic agent eluting stent (DES) designs, such as those disclosed in U.S. Pat. No. 5,871,535 and U.S. Pat. Pub. No. 2008/0233168 can be used according to the present disclosure. Stents are generally deployed using catheters having the stent attached to an inflatable balloon at the catheter's distal end. The catheter is inserted into an artery and guided to the deployment site. Once positioned at the treatment site the stent is deployed. The balloon expands the stent gently compressing it against the arterial lumen clearing the vascular occlusion or stabilizing the plaque. The catheter is then removed and the stent remains in place permanently. In many cases the catheter is inserted into the femoral artery or of the leg or carotid artery and the stent is deployed deep within the coronary vasculature at an occlusion site.

Stents, such as vascular stents, are flexible, expandable, and physically stable. Many different materials can be used to fabricate a stent used to deliver the one or more therapeutic agents according to the present disclosure. These include stainless steel, nitinol, aluminum, chromium, titanium, ceramics, and a wide range of plastics, elastomers, and natural materials including collagen, fibrin, and plant fibers. Exemplary polymeric materials include polyvinylchlorides (PVC), polycarbonates (PC), polyurethanes (PU), polypropylenes (PP), polyethylenes (PE), silicones, polyesters, polymethylmethacrylate (PMMA), hydroxyethylmethacrylate, N-vinyl pyrrolidones, fluorinated polymers such as polytetrafluoroethylene, polyamides, polystyrenes, copolymers or mixtures of these polymers.

A carrier for the one or more therapeutic agents can be associated with an intravascular treatment device (e.g., as a coating on a stent or an angioplasty balloon). The carrier can be made of one or more synthetic organic polymers, natural organic polymers, inorganics, or combinations (e.g., copolymers, mixtures, blends, layers, complexes, etc.) of these. The polymers may be biodegradable or non-biodegradable, or combinations thereof.

In certain embodiments, polymers used in accordance with teachings of the present disclosure provide biocompatible coatings for intravascular treatment devices intended for use in hemodynamic environments. In one embodiment of the present disclosure, vascular stents can be coated using a polymer composition as described herein below. Vascular stents are chosen for exemplary purposes only. Those skilled in the art of material science and intravascular treatment devices will realize that the one or more therapeutic agents described herein are useful in coating a large range of intravascular treatment devices. Therefore, the use of the vascular stent as an exemplary embodiment is not intended as a limitation.

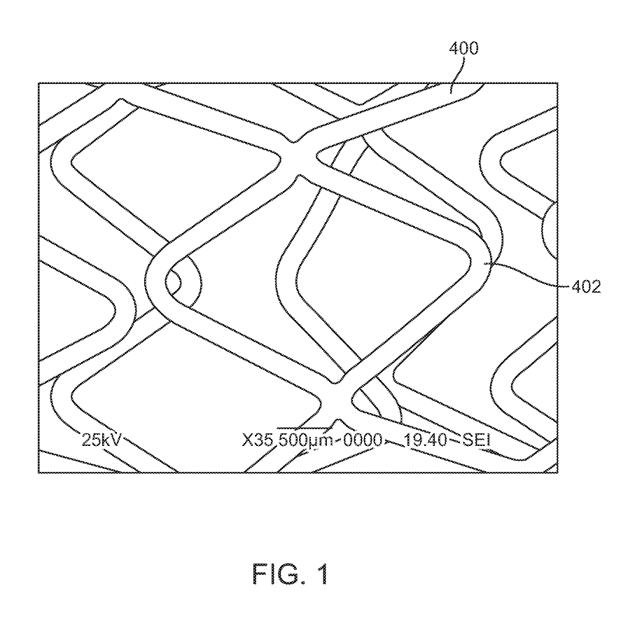

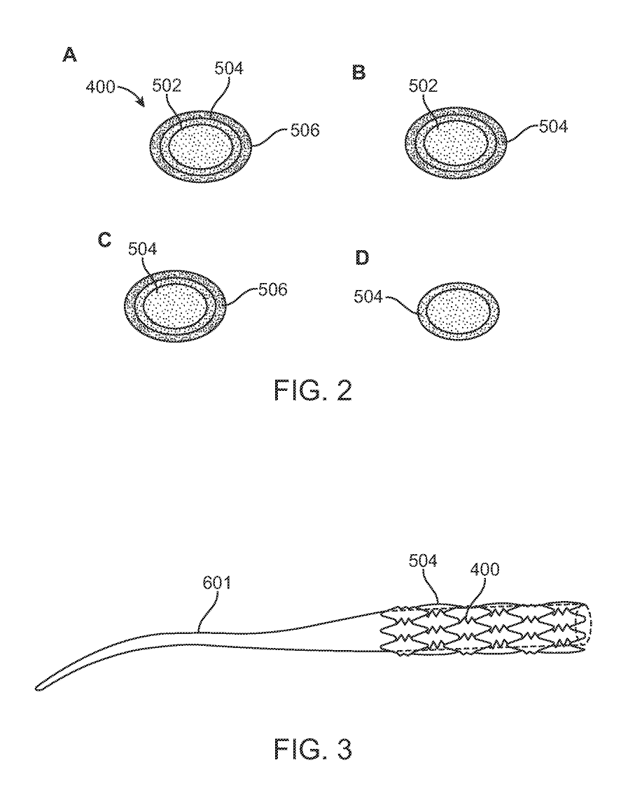

One embodiment of the present disclosure is depicted in FIG. 1. In FIG. 1 a vascular stent 400 having the structure 402 is made from a material selected from the non-limiting group materials including stainless steel, nitinol, aluminum, chromium, titanium, ceramics, and a wide range of plastics and natural materials including collagen, fibrin and plant fibers. The structure 402 is provided with a coating of one or more therapeutic agents disposed thereon, optionally with a polymeric carrier. FIG. 2 depicts a vascular stent 400 having a coating 504 made in accordance with the teachings of the present disclosure mounted on a balloon catheter 601.

FIG. 2a-d are cross-sections of stent 400 showing various coating configurations. In FIG. 2a stent 400 has a first polymer coating 502 comprising a medical grade primer, such as parylene or a parylene derivative, a second coating 504 containing one or more therapeutic agents, and a third barrier, or cap, coat 506. In FIG. 2b stent 400 has a first polymer coating 502 comprising a medical grade primer, such as parylene or a parylene derivative, and a second coating 504 containing one or more therapeutic agents. In FIG. 2c stent 400 has a first coating 504 containing one or more therapeutic agents, and a second barrier, or cap, coat 506. In FIG. 2d stent 400 has only a coating 504 containing one or more therapeutic agents. The coating 504 in each of these embodiments, may include a carrier, such as a polymeric carrier, and/or may include excipients or enhancers.

FIG. 3 depicts a vascular stent 400 having a coating 504 of the present disclosure mounted on a balloon catheter 601. A coating or one or more therapeutic agents (optionally with a carrier, e.g., to form a controlled release coating) can be applied to intravascular treatment device surfaces, either primed or bare, in any manner known to those skilled in the art. Methods compatible with the present disclosure include, but are not limited to, spraying, dipping, brushing, vacuum-deposition, and others. Moreover, a coating of one or more therapeutic agents of the present disclosure may be used with a cap coat. A cap coat as used herein refers to the outermost coating layer applied over another coating. For example, a metal stent has a parylene primer coat applied to its bare metal surface. Over the primer coat a therapeutic agent-releasing terpolymer coating or blend of homopolymer, copolymer, and terpolymer coating is applied. Over the terpolymer, a polymer cap coat is applied. The cap coat may optionally serve as a diffusion barrier to further control the therapeutic agent release, or provide a separate therapeutic agent. The cap coat may be merely a biocompatible polymer applied to the surface of the stent to protect the stent and have no effect on elusion rates.

The dilatation balloon of balloon catheter 601 shown in FIG. 3 can be used without a stent but with one or more therapeutic agents described herein disposed thereon in angioplasty procedures. For example, in the technique of Percutaneous Transluminal Coronary Angioplasty (PTCA), a dilatation balloon catheter is used to enlarge or open an occluded blood vessel which is partially restricted or obstructed due to the existence of a hardened stenosis or buildup within the vessel. This procedure requires that a balloon catheter be inserted into the patient's body and positioned within the vessel so that the balloon, when inflated, will dilate the site of the obstruction or stenosis so that the obstruction or stenosis is minimized, thereby resulting in increased blood flow through the vessel. Often, however, a stenosis requires treatment with multiple balloon inflations. Additionally, many times there are multiple stenoses within the same vessel or artery. Such conditions require that either the same dilatation balloon must be subjected to repeated inflations, or that multiple dilatation balloons must be used to treat an individual stenosis or the multiple stenoses within the same vessel or artery. Additionally, balloons and medical devices incorporating those balloons may also be used to administer one or more therapeutic agents to patients.

Balloon catheters traditionally comprise a dilatation balloon at their distal end. Angioplasty balloons are currently produced by a combination of extrusion and stretch blow molding. The extrusion process is used to produce the balloon tubing, which essentially serves as a pre-form. This tubing is subsequently transferred to a stretch blow-molding machine capable of axially elongating the extruded tubing. U.S. Pat. No. 6,328,710 discloses such a process, in which tubing pre-form is extruded and blown to form a balloon. U.S. Pat. No. 6,210,364, U.S. Pat. No. 6,283,939, and U.S. Pat. No. 5,500,180 disclose a process of blow-molding a balloon, in which a polymeric extrudate is simultaneously stretched in both radial and axial directions. Dilatation balloons are subsequently attached to a catheter shaft and wrapped down tightly on this shaft in order to achieve a low profile at the distal end of the catheter. The low profile serves to enhance the ability of a dilatation catheter to navigate narrow lesions.

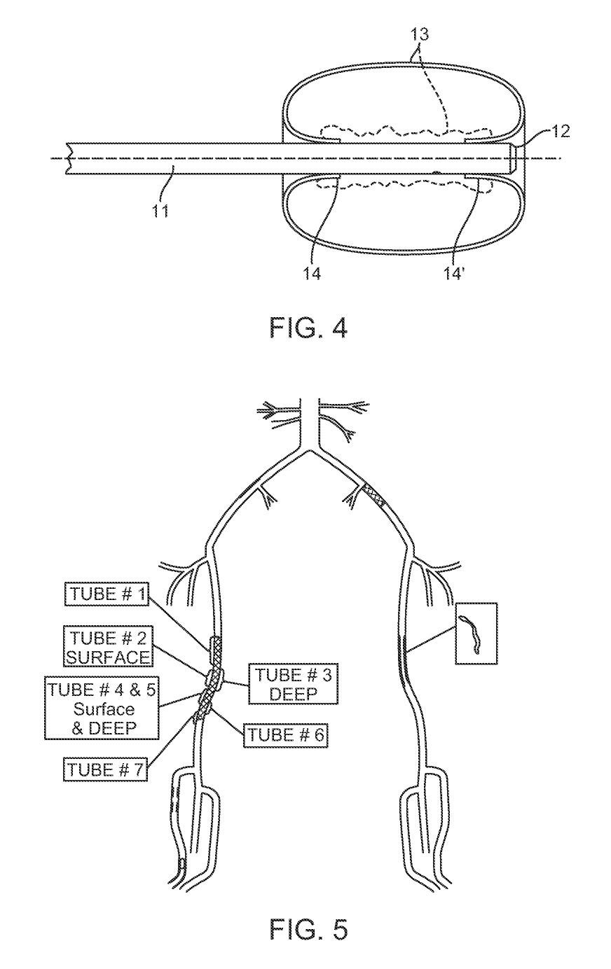

The basic design of dilatation balloons has remained, essentially, unchanged since conception. The materials used in balloons for dilatation are primarily thermoplastics and thermoplastic elastomers such as polyesters and their block co-polymers, polyamides and their block co-polymers and polyurethane block co-polymers. U.S. Pat. No. 5,290,306 discloses balloons made from polyesterether and polyetheresteramide copolymers. U.S. Pat. No. 6,171,278 discloses balloons made from polyether-polyamide copolymers. U.S. Pat. No. 6,210,364, U.S. Pat. No. 6,283,939, and U.S. Pat. No. 5,500,180 disclose balloons made from polyurethane block copolymers. Other angioplasty balloons are disclosed in U.S. Pat. No. 7,879,270, for example. An exemplary catheter (11) with a dilatation balloon is shown in FIG. 4. In this embodiment, the catheter (11) has a distal inflatable balloon (13) made up of a flexible material and having two legs (14, 14′) for its clamping on the catheter (11), wherein said legs (14, 14′) are turned inside into the balloon (13) and the balloon length between said legs (14, 14′), when expanded, extends until the catheter tip (12) or distally from that.

Elution over a prolonged time frame to inhibit the restenosis phenomenon can be used in certain embodiments; however, in certain embodiments this is neither necessary nor desirable. In certain embodiments, it is sufficient to have a time limited contact between therapeutic agent and vessel surface, for example, from a few seconds to one minute. These are typically the contact times of a catheter balloon. For example, U.S. Pat. Pub. No. WO 02/076509 discloses one or more therapeutic agent-coated catheter balloons releasing such one or more therapeutic agent in an immediately bioavailable form during the short contact time of the balloon with the vessel wall.

Prolonged therapeutic agent elution can be obtained by various solutions, such as, for example, incorporation of the one or more therapeutic agents in a polymeric matrix or microcapsules. Immediate release can also be accomplished and typically depends on several factors, of which the main ones are: the nature of the one or more therapeutic agents, in particular the hydrophilicity or hydrophobicity thereof; the form in which the one or more therapeutic agents is administered, in particular, the crystalline or amorphous form thereof; the presence of possible excipients or “enhancers” (e.g., urea); and the nature of the balloon surface on which the one or more therapeutic agents is deposited.

It should be understood that the one or more therapeutic agents typically has to be, first of all, released from the balloon to the vessel wall in the very short contact time available during an angioplasty procedure. Once the one or more therapeutic agents have been released, it is absorbed by the cell wall, before the blood flow washes it off. Ideally, it is therefore desirable that the one or more therapeutic agents absorption occurs concomitantly to the release thereof from the balloon. However, it is just as necessary that the one or more therapeutic agents are retained by the balloon surface in a manner sufficient to resist to all the handling operations to which it is subjected, both during the production step and during the preparation and carrying out of the angioplasty procedure, in any case, before the balloon reaches the site of intervention.

A coating method can include a balloon wetting step that includes, for example, dipping the balloon into a solution of one or more therapeutic agents (optionally including one or more carrier materials and/or one or more excipients or enhancers), spraying such solution onto a balloon, or depositing such solution on the balloon by means of a syringe, a micropipette, or other similar dispensing device. The balloon can be wetted with such solution in a deployed and inflated condition, or in a folded condition (e.g., with 3-6 folds). Such solution penetrates by capillarity under the folds, so as to form a depot which remains protected during the introduction step of the folded balloon into the blood vessel by means of the catheter, until reaching the site of intervention and the inflation thereof. Methods are also known to selectively coat the area under the balloon folds, leaving the outer surface substantially free from a therapeutic agent. Such methods can comprise, for example, the introduction into the balloon folds of a cannula bearing a series of micro-nozzles, through which a solution of one or more therapeutic agents is deposited on the inner surface of the folds. Such a method is described, for example, in US Pat. Pub. No. 2010/0233228. In general, independently from the method used, it is possible to repeat several times the balloon wetting step with the solution, as a function of the therapeutic agent amount which is intended to be deposited.

### Optional Therapeutic Agent Carrier

One or more therapeutic agents are localized to (adjacent or within) the site of build-up of atherosclerotic plaque. Preferably, this occurs by the placement of an intravascular treatment device that is comprised of, or within which is provided, the one or more therapeutic agents. The one or more therapeutic agents can be delivered by an intravascular treatment device as described herein in any of a variety of ways, several of which are described above. The one or more therapeutic agents can be incorporated directly into an intravascular treatment device (e.g., incorporated into a polymer for forming a graft of a stent graft), or into a carrier associated with an intravascular treatment device (e.g., as a coating on a stent or an angioplasty balloon), or coated or otherwise disposed on an intravascular treatment device without a carrier, or combinations thereof.

The one or more therapeutic agents can be mixed with, incorporated within, encased or enclosed within, a therapeutic agent carrier that can be made of one or more synthetic organic polymers, natural organic polymers, inorganics, or combinations (e.g., copolymers, mixtures, blends, layers, complexes, etc.) of these. The polymers may be biodegradable or non-biodegradable. The therapeutic agent/carrier formulation can be in the form of a film, sheet, threads, fibers (e.g., such as those used in making a graft material of a stent graft), coating (e.g., such as could be applied to a stent or angioplasty balloon), slab, gel, paste, capsule, microparticles, nanoparticles, or combinations of these. In certain embodiments, the one or more therapeutic agents are delivered by the intravascular treatment device over time to the local tissue. The carrier can be in a time-release formulation.

Protection of the therapeutic agents can also occur through the use of an inert molecule (e.g., in a cap- or over-coating over the therapeutic agents) that prevents access to the one or more therapeutic agents. For example, a coating of the one or more therapeutic agents can be over-coated readily with an enzyme, which causes either release of the therapeutic agents or activates the therapeutic agents. Alternating layers of a therapeutic coating with a protective coating may enhance the time-release properties of the coating overall. Thus, in certain embodiments, the treatment device can include least two therapeutic coatings, wherein each therapeutic coating is separated by a second coating.

The therapeutic agent/carrier formulation is preferably adapted to exhibit a combination of physical characteristics such as biocompatibility, and, in some embodiments, biodegradability and bio-absorbability, while providing a delivery vehicle for release of the one or more therapeutic agents that aid in the treatment of atherosclerotic tissue. For example, the formulation is preferably biocompatible such that it results in no induction of inflammation or irritation when implanted, degraded or absorbed.

Biodegradable materials include synthetic polymers such as polyesters, polyanhydrides, poly(ortho)esters, poly(butyric acid), tyrosine-based polycarbonates, poly(ester amide)s such as based on 1,4-butanediol, adipic acid, and 1,6-aminohexanoic acid, poly(ester urethane)s, poly(ester anhydride)s, poly(ester carbonate)s such as tyrosine-poly(alkylene oxide)-derived poly(ether carbonate)s, polyphosphazenes, polyarylates such as tyrosine-derived polyarylates, poly(ether ester)s such as, poly(epsilon-caprolactone)-block-poly(ethylene glycol)) block copolymers, and poly(ethylene oxide)-block-poly(hydroxy butyrate) block copolymers.

Biodegradable polyesters, include, for example, poly(glycolic acid) (PGA), poly(lactic acid) (PLA), poly(glycolic-co-lactic acid) (PGLA), poly(1,4dioxanone), poly(caprolactone) (PCL), poly(3-hydroxybutyrate) (PHB), poly(3-hydroxyvalerate) (PHV), poly(hydroxy butyrate-co-hydroxy valerate), poly(lactide-co-caprolactone) (PLCL), poly(valerolactone) (PVL), poly(tartronic acid), poly(beta-malonic acid), poly(propylene fumarate) (PPF) (preferably photo cross-linkable), poly(ethylene glycol)/poly(lactic acid) (PELA) block copolymer, poly(L-lactic acid-epsilon-caprolactone) copolymer, poly(trimethylene carbonate), poly(butylene succinate), and poly(butylene adipate).

Biodegradable polyanhydrides include, for example, poly[1,6-bis(carboxyphenoxy)hexane], poly(fumaric-co-sebacic)acid or P(FA:SA), and such polyanhydrides used in the form of copolymers with polyimides or poly(anhydrides-co-imides) such as poly-[trimellitylimidoglycine-co-bis(carboxyphenoxy)hexane], poly[pyromellitylimidoalanine-co-1,6-bis(carboph-enoxy)-hexane], poly[sebacic acid-co-1,6-bis(p-carboxyphenoxy)hexane] or P(SA:CPH), poly[sebacic acids co-1,3-bis(p-carboxyphenoxy)propane] or P(SA:CPP), and poly(adipic anhydride).

Biodegradable materials include natural polymers and polymers derived therefrom, such as albumin, alginate, casein, chitin, chitosan, collagen, dextran, elastin, proteoglycans, gelatin and other hydrophilic proteins, glutin, zein and other prolamines and hydrophobic proteins, starch and other polysaccharides including cellulose and derivatives thereof (such as methyl cellulose, ethyl cellulose, hydroxypropyl cellulose, hydroxy-propyl methyl cellulose, hydroxybutyl methyl cellulose, carboxymethyl cellulose, cellulose acetate, cellulose propionate, cellulose acetate butyrate, cellulose acetate phthalate, cellulose acetate succinate, hydroxypropylmethylcellulose phthalate, cellulose triacetate, cellulose sulphate), poly-1-lysine, polyethylenimine, poly(allyl amine), polyhyaluronic acids, alginic acid, chitin, chitosan, chondroitin, dextrin or dextran), and proteins (such as albumin, casein, collagen, gelatin, fibrin, fibrinogen, hemoglobin).

Non-degradable (i.e., biostable) polymers include polyolefins such as polyethylene, polypropylene, polyurethanes, fluorinated polyolefins, such as polytetrafluorethylene, chlorinated polyolefins such as poly(vinyl chloride), polyamides, acrylate polymers such as poly(methyl methacrylate), acrylamides such as poly(N-isopropylacrylamide), vinyl polymers such as poly(N-vinylpyrrolidone), poly(vinyl alcohol), poly(vinyl acetate), and poly(ethylene-co-vinylacetate), polyacetals, polycarbonates, polyethers such as based on poly(oxyethylene) and poly(oxypropylene) units, aromatic polyesters such as poly(ethylene terephthalate) and poly(propylene terephthalate), poly(ether ether ketone)s, polysulfones, silicone rubbers, epoxies, and poly(ester imide)s.

Representative examples of inorganics include hydroxyapatite, tricalcium phosphate, silicates, montmorillonite, and mica.

Preferred biodegradable polymers include polymers of lactide, caprolactone, glycolide, trimethylene carbonate, p-dioxanone, gamma-butyrolactone, or combinations thereof in the form of random or block copolymers. Preferred non-biodegradable polymers include polyesters, polyamides, polyurethanes, polyethers, vinyl polymers, and combinations thereof.

Particularly preferred polymers include the following: a polymer with phosphoryl choline functionality to encourage ionic interactions, including but not limited to methacrylate copolymer with MPC comonomer (Formula I); a polymer with multiple hydroxyl groups encouraging hydrogen bonding interaction with the therapeutic agents, including but not limited to that shown in Formula II; a polymer with acidic or basic groups encouraging acid-base interaction with the therapeutic agents, including but not limited to those shown in Formulas III and IV.

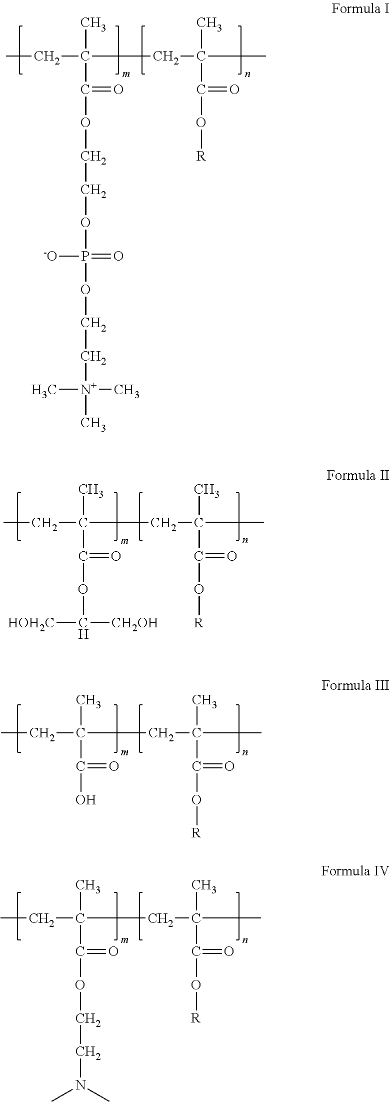

In the above formulas (I through IV), the R groups are independently C1 to C20 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to C10 substituted heteroatom substituted heteroaryl. In certain embodiments, m and n are individually integers from 1 to 20,000. In certain embodiments, m is an integer ranging from 10 to 20,000; from 50 to 15,000; from 100 to 10,000; from 200 to 5,000; from 500 to 4,000; from 700 to 3,000; or from 1000 to 2000. In certain embodiments, m is an integer ranging from 10 to 20,000; from 50 to 15,000; from 100 to 10,000; from 200 to 5,000; from 500 to 4,000; from 700 to 3,000; or from 1000 to 2000.

Particularly preferred polymers are shown below in Formulas V and VI:

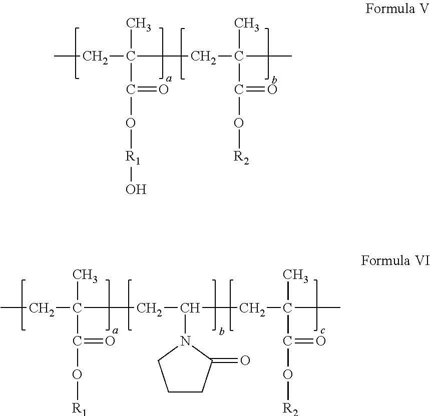

In the above formulas V, the R1 groups are independently C1 to C20 straight chain alkylene, C3 to C8 cycloalkylene, C2 to C20 alkenylene, C2 to C20 alkynylene, C2 to C14 heteroatom substituted alkylene, C2 to C14 heteroatom substituted cycloalkylene, C4 to C10 substituted arylene, or C4 to C10 substituted heteroatom substituted heteroarylene. In the above formulas V, the R2 groups are independently C1 to C20 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to C10 substituted heteroatom substituted heteroaryl. In certain embodiments, a is an integer ranging from 10 to 20,000; from 50 to 15,000; from 100 to 10,000; from 200 to 5,000; from 500 to 4,000; from 700 to 3,000; or from 1000 to 2000. In certain embodiments, b is an integer ranging from 10 to 20,000; from 50 to 15,000; from 100 to 10,000; from 200 to 5,000; from 500 to 4,000; from 700 to 3,000; or from 1000 to 2000.

In the above formula VI, the R1 and R2 groups are independently C1 to C20 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to C10 substituted heteroatom substituted heteroaryl. In certain embodiments, a is an integer ranging from 10 to 20,000; from 50 to 15,000; from 100 to 10,000; from 200 to 5,000; from 500 to 4,000; from 700 to 3,000; or from 1000 to 2000. In certain embodiments, b is an integer ranging from 10 to 20,000; from 50 to 15,000; from 100 to 10,000; from 200 to 5,000; from 500 to 4,000; from 700 to 3,000; or from 1000 to 2000. In certain embodiments, c is an integer ranging from 10 to 20,000; from 50 to 15,000; from 100 to 10,000; from 200 to 5,000; from 500 to 4,000; from 700 to 3,000; or from 1000 to 2000.

There are many polymer systems that can be used in delivering the one or more therapeutic agents described herein. Suitable examples are described, for example, in U.S. Pat. Pub. Nos. 2006/0275340 (Udipi et al.) and 2005/0084515 (Udipi et al.). Other examples of polymer systems include phosphorylcholine materials as described in U.S. Pat. No. 5,648,442 (Bowers et al.). U.S. Pat. Pub. Nos. 2006/0275340 (Udipi et al.) and 2005/0084515 (Udipi et al.) describe miscible polymer blends. Swellabilities of the miscible polymer blends are used as a factor in determining the combinations of polymers for a particular therapeutic agent.

The polymer(s) used may be obtained from various chemical companies known to those with skill in the art. However, because of the presence of unreacted monomers, low molecular weight oligomers, catalysts, and other impurities, it may be desirable (and, depending upon the materials used, may be necessary) to increase the purity of the polymer used. The purification process yields polymers of better-known, purer composition, and therefore increases both the predictability and performance of the mechanical characteristics of the coatings. The purification process will depend on the polymer or polymers chosen. Generally, in the purification process, the polymer is dissolved in a suitable solvent. Suitable solvents include (but are not limited to) methylene chloride, ethyl acetate; chloroform, and tetrahydrofuran. The polymer solution usually is then mixed with a second material that is miscible with the solvent, but in which the polymer is not soluble, so that the polymer (but not appreciable quantities of impurities or unreacted monomer) precipitates out of solution. For example, a methylene chloride solution of the polymer may be mixed with heptane, causing the polymer to fall out of solution. The solvent mixture then is removed from the copolymer precipitate using conventional techniques.

In certain embodiments described herein, the therapeutic agent/carrier formulation comprises a material to ensure the controlled release of the therapeutic agent(s). The materials to be used for such a formulation—as well as the delivery vehicle itself, in some embodiments—are preferably comprised of a biocompatible polymer, in which the one or more therapeutic agents are present. A dispersion of a therapeutic agent in a carrier, for example, allows the therapeutic reaction to be substantially localized so that overall dosages to the individual can be reduced, and undesirable side effects caused by the action of the agent in other parts of the body are minimized. The carrier can be in the form of a polymer coating, for example.

The therapeutic agents may be linked by occlusion in the matrices of the polymer coating, bound by covalent linkages to the coating or to a biodegradable stent, or encapsulated in microcapsules that are associated with the stent and are themselves biodegradable.

In certain embodiments, the therapeutic agent/carrier formulation is formulated to deliver the therapeutic agents over a period of several hours, days, or, months. For example, “quick release” or “burst” coatings are provided that release greater than 10%, 20%, or 25% (w/v) of the therapeutic agents over a period of 7 to 10 days. Within other embodiments, “slow release” therapeutic agents are provided that release less than 10% (w/v) of a therapeutic agent over a period of 7 to 10 days. Further, the therapeutic agents of the present disclosure preferably should be stable for several months and capable of being produced and maintained under sterile conditions.

In certain embodiments, therapeutic coatings may be fashioned in any thickness ranging from about 50 nm to about 3 mm, depending upon the particular use. Alternatively, such compositions may also be readily applied as a “spray”, which solidifies into a film or coating. Such sprays may be prepared from microspheres of a wide array of sizes, including for example, from 0.1 micron to 3 microns, from 10 microns to 30 microns, and from 30 microns to 100 microns.

The therapeutic agents of the present disclosure also may be prepared in a variety of “paste” or gel forms. For example, within one embodiment of the disclosure, therapeutic coatings are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” readily may be made utilizing a variety of techniques. Other pastes may be applied as a liquid, which solidify in vivo due to dissolution of a water-soluble component of the paste.

In other embodiments, the therapeutic compositions of the present disclosure may be formed as a film. Preferably, such films are generally less than 5, 4, 3, 2, or 1 mm thick, more preferably less than 0.75 mm, 0.5 mm, 0.25 mm, or, 0.10 mm thick. Films can also be generated of thicknesses less than 50 microns, 25 microns or 10 microns. Such films are preferably flexible with a good tensile strength (e.g., greater than 50, preferably greater than 100, and more preferably greater than 150 or 200 N/cm²), have good adhesive properties (i.e., adhere to moist or wet surfaces), and have controlled permeability.

### EXAMPLES

Objects and advantages of this disclosure are further illustrated by the following examples, but the particular materials and amounts thereof recited in these examples, as well as other conditions and details, should not be construed to unduly limit this disclosure.

### Results and Discussion

The aim of MAPA study was to better understand atherosclerosis and post-interventional restenosis in peripheral vascular disease. Of a particular interest was the superficial femoral artery (SFA) given that it is the most prominent location for intervention with a high rate of unresolved complications and recurring stenosis. Moreover drug-eluting stents (DES) which reduced the rate of restenosis in coronary arteries down to nominal single digits have not demonstrated comparable success in the treatment of SFA.

In order to gain an understanding for the development of restenosis in SFA lesions we obtained pertinent disease specimens that were collected during atherectomy procedures. The collected samples were studied through comparison between various disease states, e.g. de novo vs. restenosis vs. non-diseased SFA. The performed analysis focus on the relative expression of genes that mark inflammation, proliferation, and production of extracellular matrix which were previously identified to play important role in the progress of atherosclerosis and the development of restenosis in coronary arteries. In addition, we performed comparative analysis of specimens obtained from the same patient at different time points due to re-occlusion of the lesion post revascularization or due to the presence of occlusive SFA disease in the other leg. Analyzing samples that originated in the same individual patient gave us the opportunity to follow progression of the disease, from de novo lesion to a lesion that has re-occluded due to restenosis (sometimes more than once).

The results from this study delineate selected genes that are being most persistently up regulated trough the development of atherosclerosis and restenosis in SFA, as well as identifying the unique genes that show unique expression pattern and are modulated with the development of restenosis.

In addition, obtaining samples from SFA arteries allowed us to generate SFA derived-smooth muscle cells and to study their response to anti-proliferative drugs that are currently in use with combination devices treating coronary and peripheral disease. These data might help in selecting the best therapeutic approach to treat atherosclerosis in SFA.

### Results and Discussion

### Demographics and Baseline Description

We analyzed 57 samples from 21 patients with SFA restenosis, 69 samples from 25 patients with de novo SFA disease, and 11 non-diseased SFA arteries. Patient characteristics are detailed in Table 1.

Generally samples from de novo and restenotic patients were of a matching age range (60-80y). The prevalence of known diabetes was high in both cohorts and not different between the groups (9 of 25 versus 10 of 21), which are consistent with the general demographics of PAD patients. The use of statins was also prevalent in both groups (19 of 25 versus 18 of 21). The revascularized patients included patients with claudication (14 of 25 versus 11 of 21) and ischemia (9 of 25 versus 7 of 21). The samples from no PAD control patients (Table 1) were from younger donors (Table 1), age range 20-45

TABLE 1
Clinical characteristics of patient cohort studied in
gene expression and histological analysis

| | Controls | De Novo | Restenotic |
|---|---|---|---|
| | (n = 12) | (n = 25) | (n = 21) |
| Age, yrs | 40 ± 15.5 | 73 ± 10.3 | 73 ± 8 |
| Sex, n (%) | | | |
| Male | 6 (50%) | 16 (64%) | 18 (86%) |
| Female | 6 (50%) | 9 (36%) | 3 (14%) |
| Diabetes, n (%) | 1 (8%) | 9 (36%) | 10 (48%) |
| Hypertension, n (%) | 6 (50%) | 19 (76%) | 14 (67%) |
| Tobacco, n (%) | 6 (50%) | 12 (48%) | 8 (38%) |
| Known CAD, n (%) | 0 (0%) | 17 (68%) | 15 (71%) |
| Statins, n (%) | 0 (0%) | 19 (76%) | 18 (86%) |
| Previous SFA | 0 (0%) | 17 (68%) | 21 (100%) |
| revascularization, n (%) | | | |
| Claudication, n (%) | 0 (0%) | 14 (56%) | 11 (52%) |
| Ischemic PAD, n (%) | 0 (0%) | 9 (36%) | 7 (33%) |

FIG. 5 shows a representative diagram of patient superficial femoral arteries and site of lesion harvest. Box insert representative of tissue specimen. (A) De novo and restenotic lesions were procured from individual patients in areas outlined in black. Additional samples were harvested from a subset of patients that returned for follow up procedures. Atherectomy samples were processed for gene expression profiling and histological analysis.

### Gene Expression Analysis

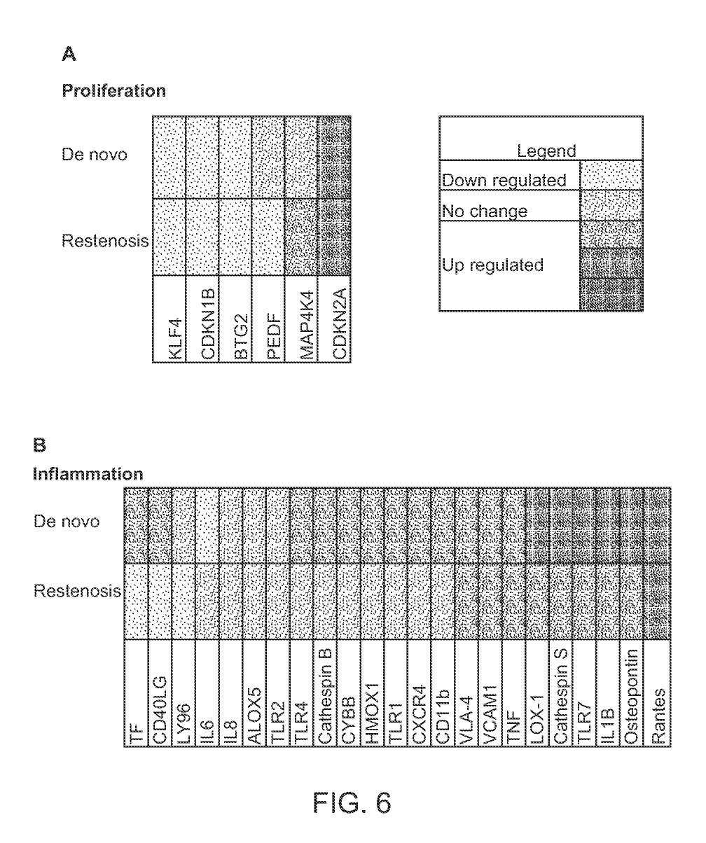

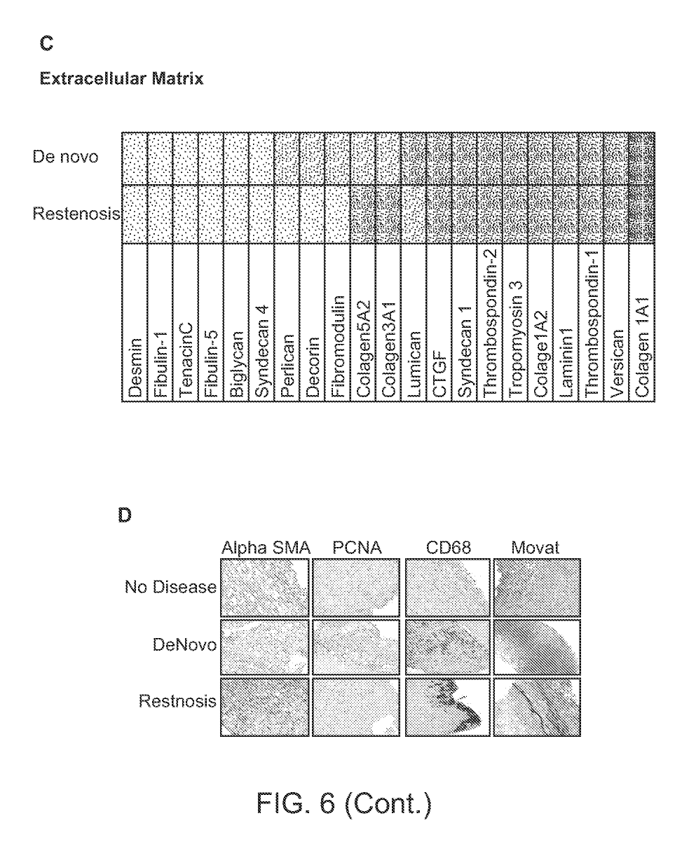

In order to gain an understanding for the development of restenosis in SFA lesions we analyzed the relative expression of the selected genes (see materials and methods for full genes list and their respective known functions) in the de novo and restenosis specimens compared to the non-diseased control samples. The comparison of the gene expression analysis is summarized as a ‘heat map’ in FIG. 6; FIG. 6A shows modulated genes that could play role in the control of cell cycle and proliferation of vascular smooth muscle cells. The data reveals that the transcriptional expression of genes that inhibit proliferation of smooth muscle cells is substantially reduced in both de novo samples (3 genes, BTG2, KLF4 and CDKN1B) as well as in the restenotic samples (4 genes, BTG2, KLF4 and CDKN1B and PEDF) relative to the non-disease controls, which served as the base-line for changes in gene expression. In addition, there is an enhancement the expression of CDKN2A gene, which is related to inhibition of apoptosis and maintenance of cell cycle in both de novo as well as in the restenotic samples.

In general, these findings suggest an enhanced proliferative state of the neointimal SMCs in both de novo and restenotic disease states and delineate PEDF for the differential expression in the restenotic samples.

The transcriptional expression of genes that are associated with vascular inflammation is shown in FIG. 6B. Out of the 23 modulated genes presented in the heat map 22 are significantly up regulated in the de novo samples, confirming the strong inflammatory makeup of the atherosclerotic disease in SFA. In a similar manner, thought to a leaser extent, restenotic samples showed substantial up regulation of the inflammatory gene expression when compared to the baseline, showing up regulation of 20 genes (out of the total of 23 modulated). Interestingly, these data also outlines one gene, the cytokine IL-6, to be differentially up regulated in the restenotic but not the de novo samples.

The modulation of gene expression associated with extracellular matrix (ECM) proteins is shown in FIG. 6C. Interestingly, majority of the modulated ECM genes presented in the hit map show similar profile between de novo and restenotic samples relative to the non-disease controls. 6 ECM genes show differential expression between de novo and restenotic samples, 5 of which are modulated in restenotic but not the de novo samples, including the down regulation of perlican, fibromodulin and decorin and the upregulation of Collagen 5A2 and Collagen 3A1.

FIG. 6D shows the immunohistochemical staining of representative specimens from the non-disease, de novo and the restenotic patients. The samples were stained for presence of smooth muscle cells (alpha SMA), for presence of proliferating smooth muscle cells (PCNA), for presence of inflammatory cells (CD68) as well for an ECM presence (Movat). The results confirm the increased presence of actively proliferating smooth muscle cells in the de novo and restenotic samples, extensive presence of inflammatory cells, and abundant presence of collagen (blue).

### Analysis of Cell Cycle Regulation and SMC Proliferation in SFA Restenotic Subjects

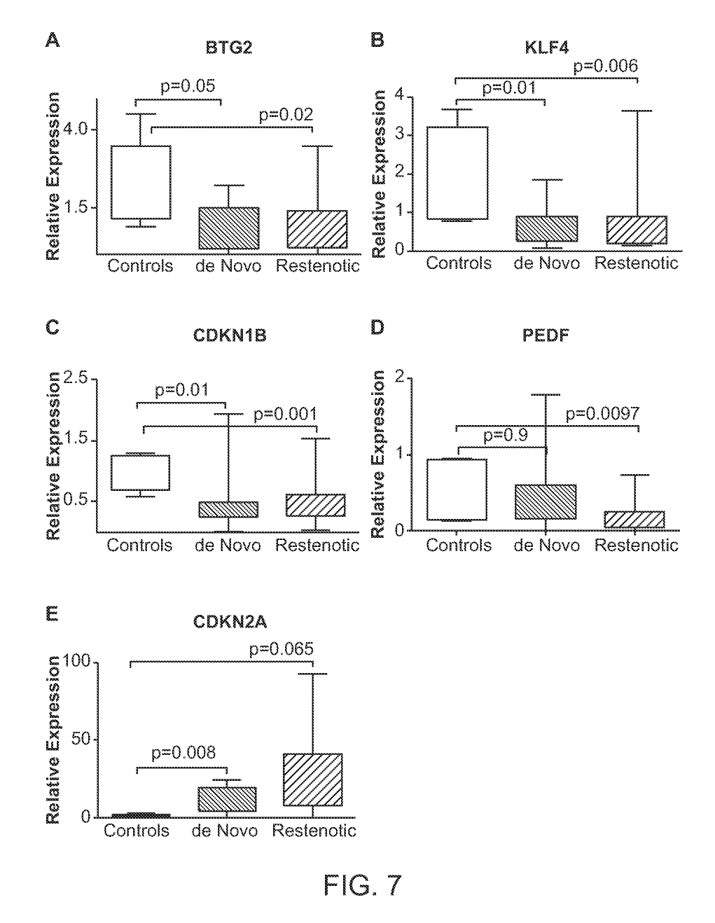

It is an accepted hypothesis that the development of restenosis post revascularization is due to activation of vascular smooth muscle cells which triggers their proliferation and subsequent production of extracellular matrix. While there is a significant amount of experimental data with reference to this process for coronary restenosis, it is less established in the context of SFA restenosis. To better understand the characteristics of proliferative activation in SFA lesions we used the samples collected from the SFA lesions and studied the expression of the most prominent known cell cycle inhibitors in the de novo and the restenotic samples. The results presented in FIG. 7 demonstrates a substantial down regulation of the cell cycle inhibitors BTG2, KLF4 and CDKN1B across both, the de novo and the restenotic samples, when compared to the non-disease controls (FIG. 7A-C). These data suggest that the smooth muscle cells in the atherosclerotic SFA lesions activated and proliferating due to removal of the cell cycle arrest as indicated by down regulation of these inhibitory molecules expression (mention not shown data-markers that were not modulated). Interestingly, we observed selective inhibition, in the restenotic but not in the de novo samples, in the expression of (PEDF) gene (FIG. 7D) that is known to inhibit proliferation. This result suggests a potential role (previously unknown) for PEDF in the proliferative activation of vascular smooth muscle cells during in the development of restenotic lesion. Interestingly, we also observed an up regulation in the expression of the regulatory cell cycle molecule CDKN2A, in de novo while to a higher extent in the restenotic samples (FIG. 7E).

These data suggests a coordinated regulation of the cell cycle in smooth muscle cells of the SFA atherosclerotic lesions, which renders them to a higher level of proliferative state. These data also delineates PEDF having a potential role in the development of restenotic lesions in SFA.In addition we performed comparative analysis of specimens obtained from the same patient at different time points due to re-occlusion of the lesions. Such analysis eliminates the variants that affect gene expression, like genetic background, drugs regiments, severity of PAD disease, co-morbidities, age, etc. Thus, investigating de novo and restenotic samples originated from individual patients allowed us to examine the consistency in the modulation of identified genes.

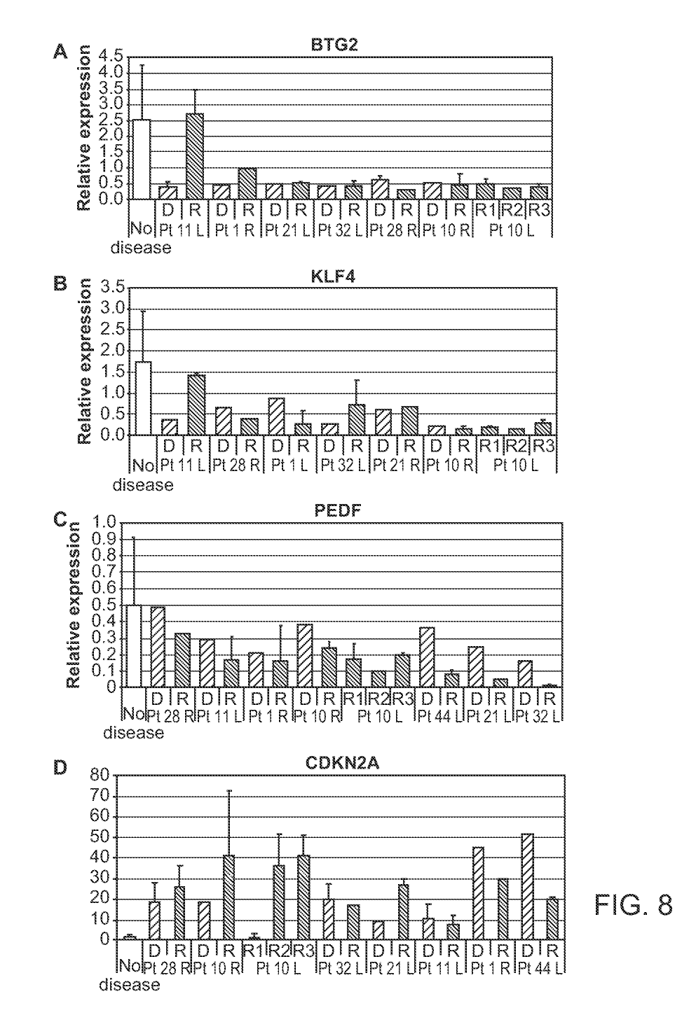

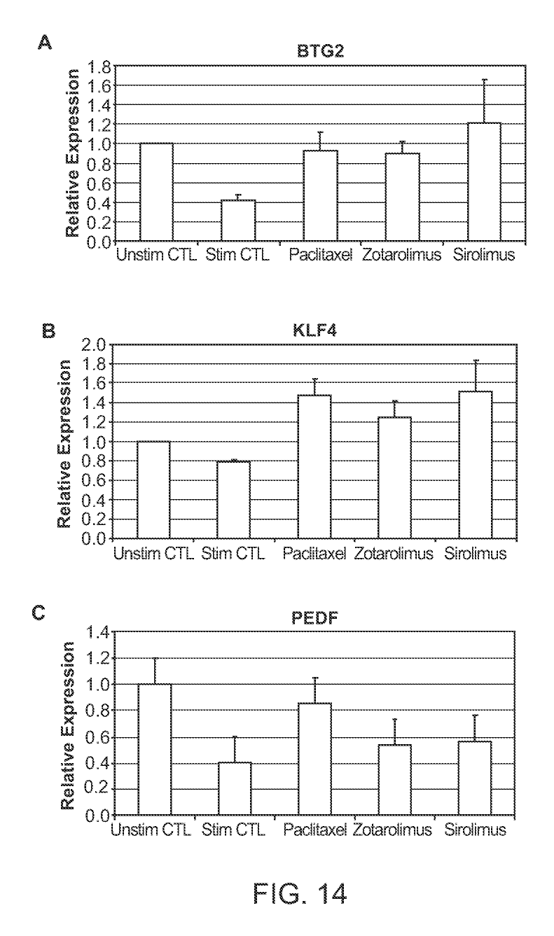

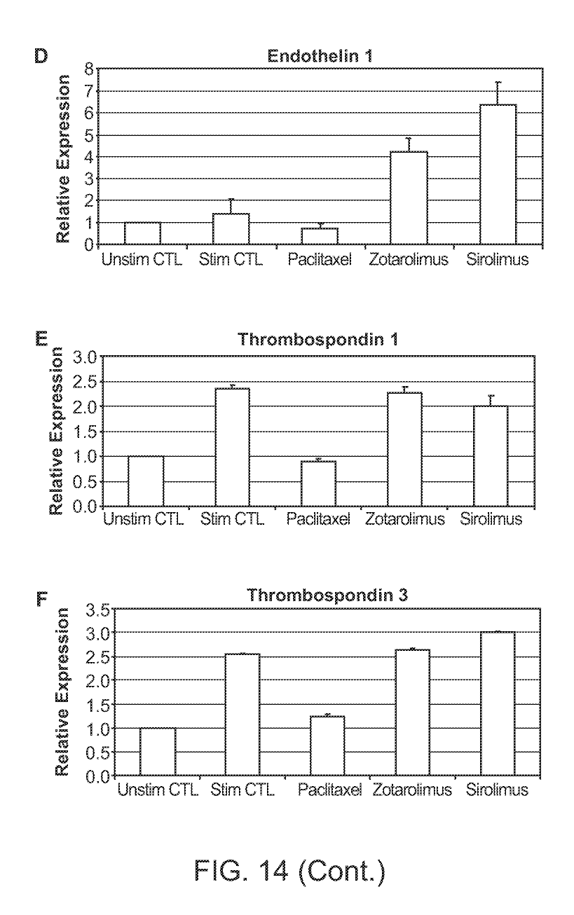

Interestingly, BTG2 and KLF4 were the most pronounced genes down regulated in both de novo and restenotic individual patients, across most of the matching samples (14 out 15 specimens for BTG2, FIGS. 8A and 14 out 15 specimens for KLF4, FIG. 8B) suggesting prominent causal association with activation of proliferative response in both de novo and restenotic disease states. In contrast, the down regulation of CDKN1B was apparent only in few of the paired patient samples (5 out of 15 specimens, data not shown) suggesting heterogeneity between various patients and thus possible heterogeneity in its causal association with SFA atherosclerosis and restenosis. Most remarkable is the selective down regulation of PEDF in all/most individual restenotic samples that were analyzed (FIG. 8C) suggesting causal association with activation of proliferative response during the development of restenosis. Also interesting is the up regulation CDKN2A that is apparent in most of the paired individual disease samples (FIG. 8D), confirming its potential involvement in the proliferative response and consistent with the data presented in FIG. 7E.

### Expression of Pro-Inflammatory Molecules in the SF Restenotic and Atherosclerotic Samples

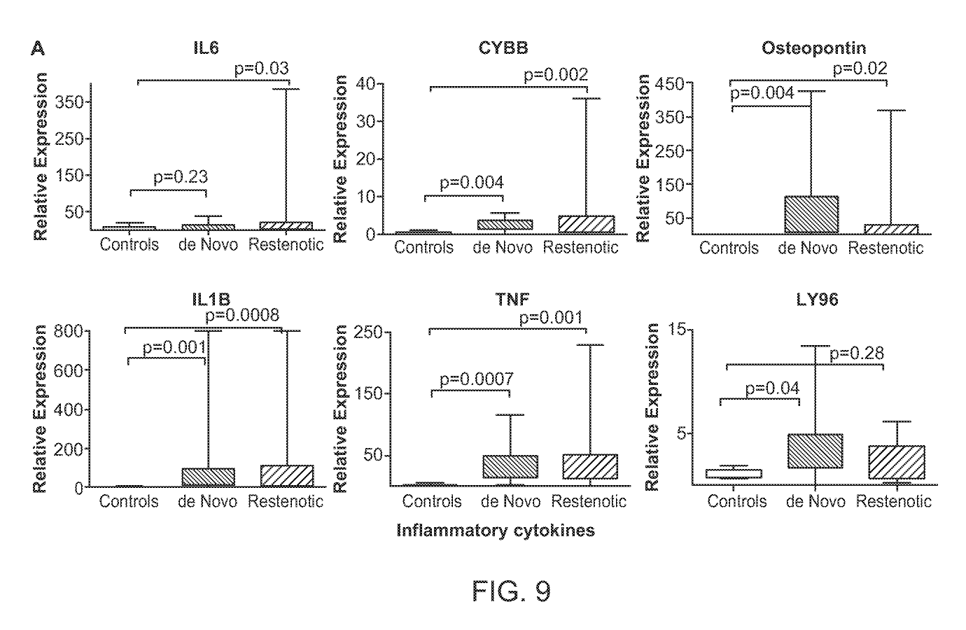

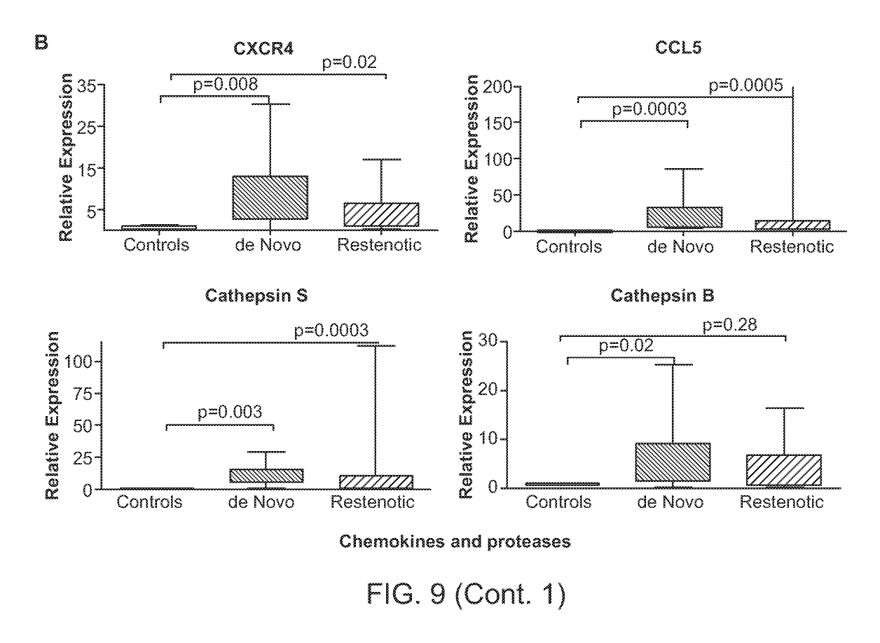

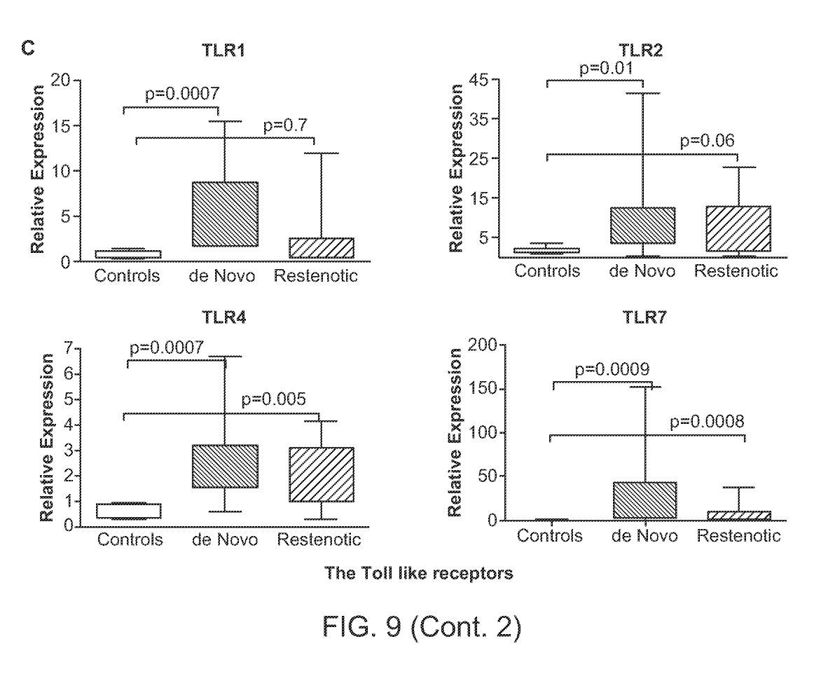

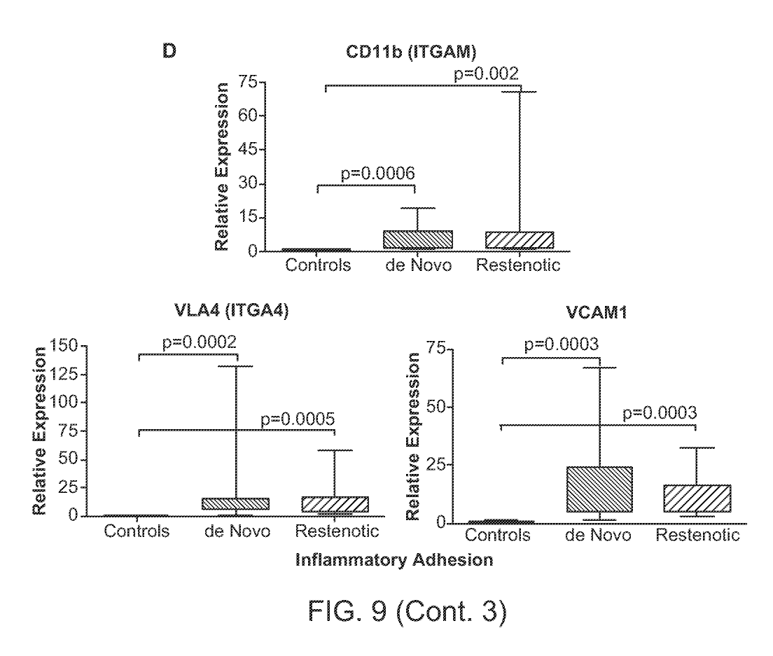

As shown in FIG. 6B, both de novo and restenotic samples showed significantly enhanced expression of various molecules that trigger and maintain vascular inflammation. Notably, majority of the inflammatory molecules are significantly up regulated in both, de novo and restenotic samples, though the magnitude of expression enhancement appears to be increased in the de novo samples. FIG. 9 shows representative genes in de novo and restenotic patients compared with the non-disease controls. It is notable that the expression of the inflammatory cytokine IL-6 (FIG. 9A) is substantially increased in the restenotic samples more than in the de novo. The expression of all the other inflammatory genes was up regulated to a greater or comparable extent in the de novo and the restenotic samples (FIGS. 9A, B and C), including the expression of inflammatory cytokines and chemokines, such as IL-1 beta, TNF, CCL5 and its receptor CXCR4. In addition, notable the up regulation of CYBB, gene that is involved in initiation of oxidative stress, and LY96, gene that is involved in development of atherosclerotic lesions, as well as of the inflammatory proteases, such as Cathepsin S and Cathepsin B. Notable also is the comprehensive up regulation of molecules from the Toll receptor pathway (TLR, FIG. 9C) that are consistently up regulated in de novo samples, across the various family members we evaluated, including TLR1, TLR2, TLR4 and TLR7. Noteworthy is also a group of specific integrins (FIG. 9D) that mediate inflammatory cell-cell interactions, in particularly monocytic adherence to vascular cells and their tissue extravasation including ITGAM (CD11b), ITGA4 (VLA4) and VCAM.

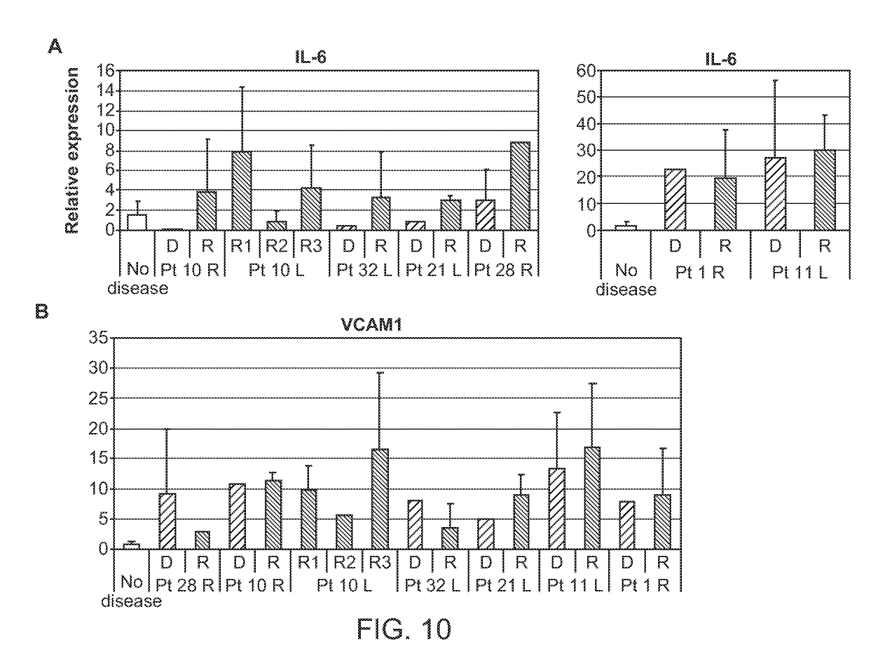

FIG. 10A shows the expression of IL-6 in specimens obtained from the same patients at a different time points. Remarkably, the paired comparison between the de novo and restenotic lesions in these patients shows consistent increase in IL6 expression from de novo to restenotic lesions suggesting that IL-6 is a prominent inflammatory component that drives the development of restenosis in SFA.

FIG. 10B shows that the expression of VCAM in these specimens is up regulated in all de novo and restenotic samples confirming the findings presented in FIG. 9D and supporting the importance of inflammatory adhesion molecules, such as VCAM, in development and progression of atherosclerosis in SFA.

### Modulation of Extra-Cellular Matrix Gene Expressions in the SFA Restenotic and Atherosclerotic Samples

As shown in FIG. 6 both, the restenotic and the de novo samples reveal a pronounced modulation of ECM gene expression, being either up or down regulated, relative to the non-disease baseline. Nevertheless, the modulation is more pronounced in the restenotic samples (20 out of 21 genes) than in the de novo (16 out of 21). Also notably, given that the atherectomy samples lack the inner layers of the artery and the control samples include it, the down regulation of some ECM genes that constitute the internal layers and the basal lamina in the atherectomy samples could be attributed to this variance.

In contrast, the up-regulation of ECM genes detected in the atherectomy samples is driven by their expression in the luminal surface, encompassing the stenotic disease. Therefore the up regulation of these genes is indicative of the inflammatory activation of vascular cells and of the disease state. In addition, the down regulation of secreted extracellular matrix proteins that have explicit function in healing or inflammation and is most likely indicative of changes related to disease state. For example, perlican (HSPG2) are down regulated.

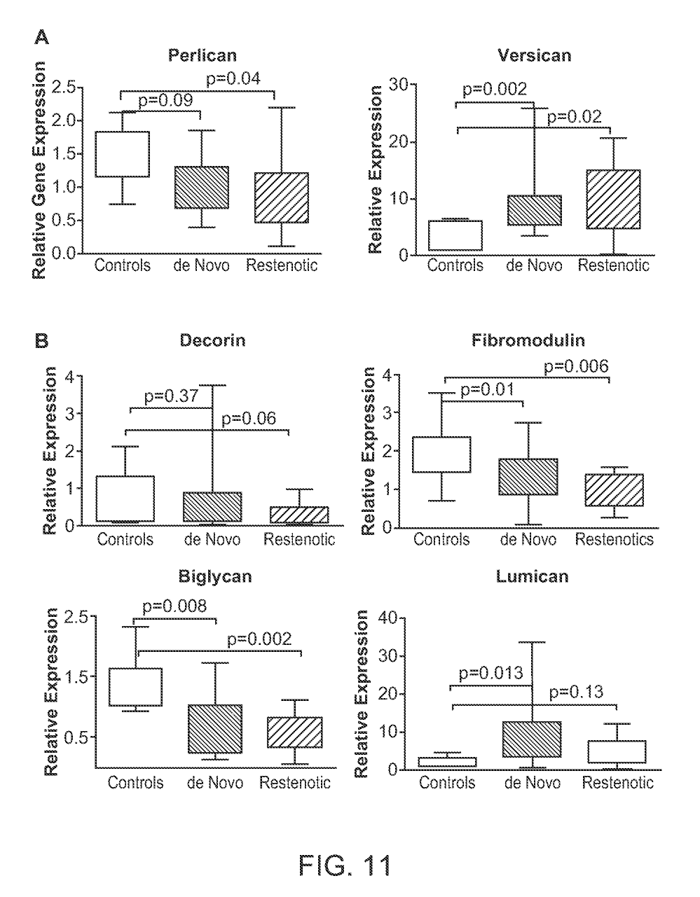

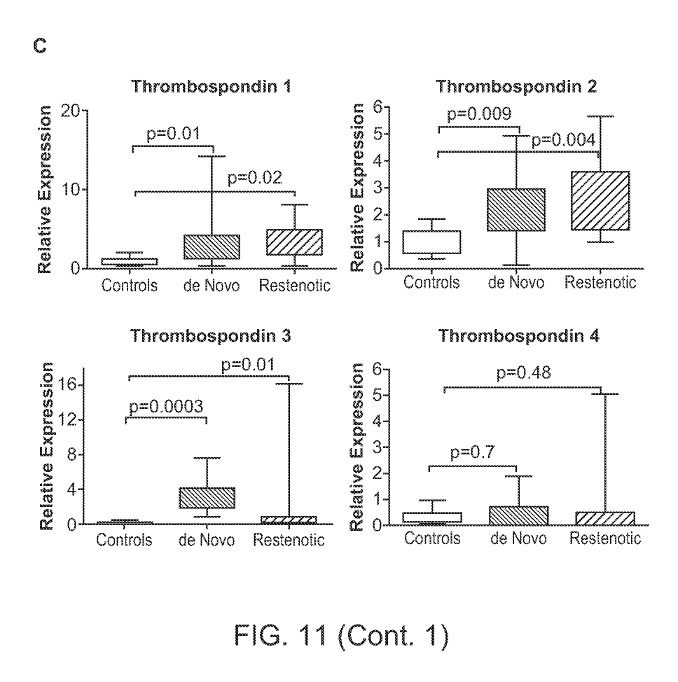

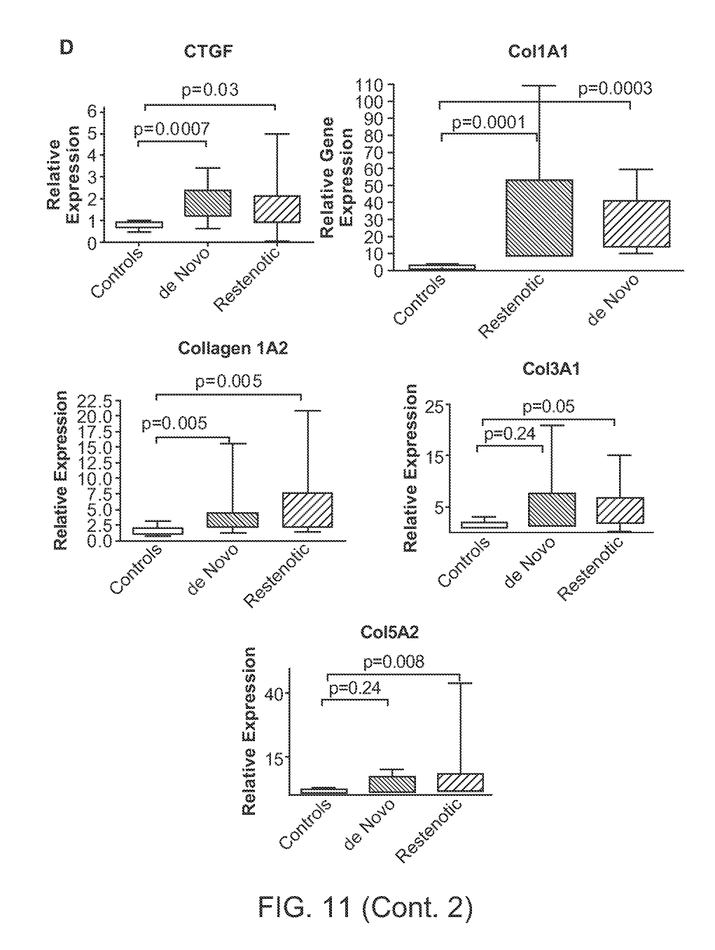

An example for modulation of such gens is shown in FIG. 11A, perlican (HSPG2), a secreted ECM protein is significantly down regulated in the restenotic as well as in the de novo samples. Perlican was extensively studied (ref) for its role in inhibition of smooth muscle cell proliferation as well as anti inflammatory function during vascular healing (ref). In agreement with this data, the expression of the ECM protein, versican is upregulated in both de novo and restenotic samples. Versican have a functional role in vascular cell adhesion and migration and it has been shown to enhance smooth muscle cell proliferation and reduce their apoptosis. Thus, the down regulation of perlican and the up regulation of versican suggesting increased inflammatory in the disease specimens. FIG. 11B shows an expression of ECM genes from the small leucine-rich proteoglycan (SLRP) family, which includes decorin, biglycan, fibromodulin and lumican, proteins that bind collagen fibrils and regulate the interfibrillar spacings. Interestingly, the expression of lumican is up regulated in the de novo and less in the restenotic samples, while decorin and fibromodulin are down regulated in both. These data suggest that while decorin and biglycan are part of the ECM that constitutes the basal layer of the artery while lumican and collagen makeup the de novo and the restenotic ECM.

An interesting finding, shown in FIG. 11C, is the differential/selective up regulation of genes from the Thrombospondin family, Thrombosponin-1, Thrombosponin-2 and Thrombosponin-3, but not of Thrombosponin-4, in a similar manner in both, de novo and the restenotic samples/specimens. These secreted multi-functional glycoproteins have been postulated to modulate cell adhesion, SMC proliferation as well as regulating angiogenesis and inflammation.

FIG. 11D shows the up regulation of CTGF, a growth factor that in response to injury triggers a coordinated expression of extracellular matrix proteins in both, de novo and restenotic samples (ref). In agreement, collagen 1A1 and collagen 3A1 are also up regulated in both, de novo and restenotic samples, and collagen 1A2 and collagen 5A2 are more significantly up regulated in the restenotic samples.

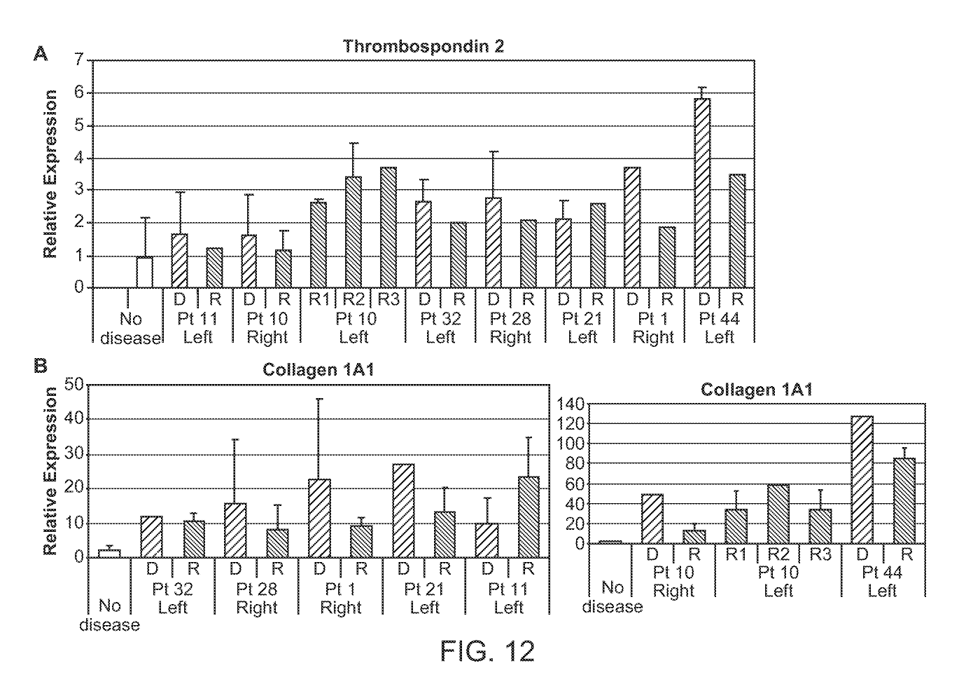

Taken together, the modulated expression of ECM in de novo and restenosis atherosclerotic disease states indicates a phenotypic shift from the normal mille of extracellular matrix (produced by healthy SMC) to an aberrant and unbalanced composition that indicate and fosters inflammatory and proliferative activation of SMC. FIG. 12 shows that the expression of Thrombospondin-2 and Collagen A1A in specimens obtained from the same patients is up regulated in most of the de novo and restenotic paired samples confirming the findings presented in FIG. 11 with regards to abnormal ECM composition in these lesions.

### Transcriptional Response to Anti-Proliferative Drugs

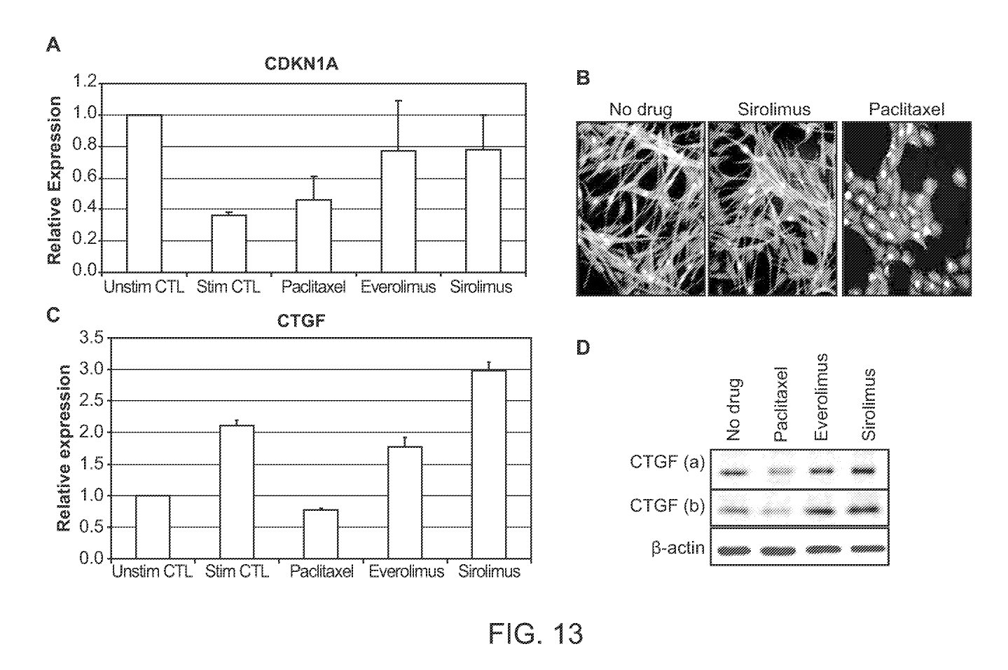

Obtaining samples from SFA arteries allowed us to generate SFA derived-smooth muscle cells and to examine the expression of genes of interest as ide12tified in the disease atherectomy samples. In particularly we investigated the transcriptional response to anti-proliferative drugs, e.g. paclitaxel and drugs from the limus family such as sirolimus, everolimus or zotarolimus. These drugs are currently employed in combination devices indicated for the treatment of coronary and peripheral disease, including drug eluting stents and drug eluting balloons. The differential mechanism of action of the anti-proliferative drugs is illustrated in FIGS. 13A and 13B, signifying that limus drugs, such as sirolimus and everolimus inhibit proliferation by affecting cellular signaling, in particularly by up regulating cell cycle inhibitors, such as CDKN1A (FIG. 13A) rendering the cells to Gₒ cell cycle arrest. Paclitaxel, on the other hand, does not affect CDKN1A expression (FIG. 13A), and arrests the cells during the cell cycle metaphase, by binding to the microtubules, disrupting cellular cytoskeleton (FIG. 13B) and preventing the cells from completing the cell division. Given the well described link between the cytoskeleton, modulation of cellular signaling and ECM regulation, we further investigated the effects of paclitaxel and the limus drugs on CTGF gene expression. FIG. 13C reveals a substantial down regulation in the expression of CTGF by paclitaxel but not by the limus drugs. In agreement with this result, the CTGF protein levels are reduced by paclitaxel but by not the limus drugs (FIG. 13D). Since we also observed that CTGF is up regulated in the disease samples from de novo and restenotic patients (FIG. 13D) its down regulation by paclitaxel may elucidate the therapeutic benefits recently observed with the paclitaxel eluting drug coated balloon angioplasty.

Since the limus drugs have been known to affect the signaling via their effect on cell cycle inhibitors (CDKN1A and CDKN1 B), we next studied their effects on the pertinent proliferative disease targets identified in this study. Specifically, we looked at BTG2, KLF4 and PEDF (FIGS. 14A, 14B, and 14C, respectively). The data reveals that inflammatory stimulation (see materials and methods for more details) of SFA derived-smooth muscle cells cause reduction in the expression of BTG2, KLF4 and PEDF, rendering the cells into more proliferative state. These data is in agreement with the substantial reduction in the levels of BTG2, KLF4 and PEDF that we observed restenotic disease samples (FIG. 8). The limus drugs, sirolimus and zotarolimus induced the expression of BTG2, KLF4 cell cycle inhibitors, but not of the proliferation PEDF inhibitor, which are previously unknown actions for these drugs. Interestingly, pacitaxel up regulated the expression of all, BTG2, KLF4 and PEDF, suggesting a novel/complementary mode of action by which paclitaxel inhibits cell division.

FIG. 14 shows additional genes of interest that are modulated by paclitaxel and the limus drugs in SFA-SMC cells; Endothelin-1 is substantially upregulated by sirolimus and zotarolimus, while slightly inhibited by paciltaxel. In a similar manner, the expression of Thrombospondin-1 and Thrombospondin-3 is inhibited by paciltaxel but not by sirolimus and zotarolimus. Taken together the data with regards to the differential effects of commercially employed, anti-proliferative drugs, such as paciltaxel and drugs of the limus family, on the expression of SFA disease target genes can highlight/point to wards the most beneficial therapeutic mode of application and treatment.

### Discussion

The aim of MAPA study was to the advance our understanding of SFA atherosclerosis and restenosis by investigating the transcriptional profile of clinical sample collected during atherectomy procedures. Foremost, the MAPA study results have demonstrated the strong inflammatory makeup of the atherosclerotic disease in SFA, revealing that the vascular inflammation underlying the de novo stenotic disease is still prevalent in the post intervention restenotic lesions; the vast up regulation of genes associated with vascular inflammation in the de novo patient specimens is sustained at large in the restenotic patient specimens. Interestingly, the data outlines the enhanced up regulation of the inflammatory cytokine IL-6 in the restenotic vs. de novo patient specimens. Moreover, the remarkable consistency in the increase of IL6 gene expression in the paired de novo and restenotic lesions from same patients might indicate that IL-6 is a prominent inflammatory component that drives the development of restenosis in SFA. Also notable is the comprehensive up regulation of molecules from the Toll receptor pathway and the up regulation of specific integrins that mediate inflammatory cell-cell interactions.

It is an accepted hypothesis that the activation of vascular smooth muscle cells post revascularization, due to the injury and inflammation, triggers their proliferation. SMC proliferation, migration and the subsequent production of extracellular matrix encompass neointimal growth leading to restenosis. While there is an ample support for the various steps of this process for coronary artery restenosis, it is less established in the context of SFA restenosis.

Our analysis of the cell cycle and proliferation profile of SFA specimens reveals an enhanced proliferative state in both de novo and restenotic disease states via substantial down regulation of the cell cycle inhibitors BTG2, KLF4 and CDKN1B compared to the non-disease controls. Comparative analysis of specimens obtained from the same patient at different time points due allowed us to examine the consistency in the modulation of identified genes. Interestingly, KLF4 and BTG2 were the most pronounced genes down regulated in both de novo and restenotic individual patients, across most of the matching samples suggesting prominent causal association with activation of proliferative response in both de novo and restenotic disease states. In contrast, the down regulation of CDKN1B was apparent only in few of the paired patient samples suggesting heterogeneity between various patients and thus possible heterogeneity in its causal association with SFA atherosclerosis and restenosis In addition, the up regulation CDKN2A was apparent in most of the paired individual disease samples, confirming its potential involvement in the proliferative response. Notably, the expression of proliferation inhibitor, PEDF, was noticeably selective for restenotic more than to de novo samples. This result was confirmed within the analysis of individual repeat patient specimens; where the selective down regulation of PEDF expression was observed in most of the paired restenotic samples, strongly suggesting a causal association between PEDF and the activation of proliferative response during the development of restenosis.

We also studied the effects of the anti-proliferative drugs, paclitaxel and drugs from the limus family on the expression of these proliferative targets in SFA derived-smooth muscle cells. Interestingly, the limus drugs induced the expression of BTG2, KLF4 cell cycle inhibitors, but not of PEDF, while pacitaxel up regulated the expression of all, BTG2, KLF4 and PEDF, suggesting a novel mode of action by which paclitaxel inhibits cell division.

The modulation of gene expression associated with extracellular matrix (ECM) is vastly pronounced in both, the restenotic and the de novo samples, being either up or down regulated, relative to the non-disease baseline.

Given that the atherectomy samples lack the inner layers of the artery and the control samples include it, the down regulation of some ECM genes that constitute the internal layers and the basal lamina in the atherectomy samples could be attributed to this variance. In contrast, the up-regulation of ECM genes detected in the atherectomy samples is driven by their expression in the luminal surface, encompassing the stenotic disease. Therefore the up regulation of these genes is indicative of the inflammatory activation of vascular cells and of the respective disease state. In addition, the down regulation of secreted extracellular matrix proteins that have explicit function in healing or inflammation and is most likely indicative of changes related to disease state, e.g. the combined down regulation of perlican, a secreted ECM protein that possess anti-inflammatory and anti proliferative functions, combined with the up regulation of versican, an anti-apoptotic and pro-proliferative ECM protein, suggests coordinated phenotypic shift indicative of increased inflammation and proliferation that is driven and supported by the altered expression of ECM milieu. In agreement with these results was the up regulation of Thrombosponin-1, Thrombosponin-2 and Thrombosponin-3 genes expression. These secreted multi-functional glycoproteins have been postulated to modulate cell adhesion, SMC proliferation as well as regulating angiogenesis and inflammation. In addition, the up regulation of CTGF expression and subsequent upregulation of collagen production is in agreement with supporting the concept with regards to the central role that modulation of ECM expression plays in both de novo and restenotic SFA disease. Notably, the expression of CTGF in SFA derived-smooth muscle cells is substantially down regulated by paclitaxel, along with the expression of Endothelin-1, Thrombospondin-1 and Thrombospondin-3. Thus, the differential effects of drugs that are utilized in combination device that treat atherosclerosis and restenosis in SFA should be taken in account when new device are evaluated for their therapeutic benefits or new combination device are designed.

The complete disclosures of all patents, patent applications, publications, and nucleic acid and protein database entries, including for example GenBank accession numbers and EMBL accession numbers that are cited herein are hereby incorporated by reference as if individually incorporated. Various modifications and alterations of this disclosure will become apparent to those skilled in the art without departing from the scope and spirit of this disclosure, and it should be understood that this disclosure is not to be unduly limited to the illustrative embodiments set forth herein.

### SEQUENCE LISTING FREE TEXT

SEQ ID NO:1 Full length amino acid sequence of PEDF
SEQ. ID NO:2 A 44-amino acid sequence region of PEDF

What is claimed is:

1. A method of treating atherosclerosis in a subject, the method comprising:
providing an intravascular treatment device comprising one or more therapeutic agents, wherein the one or more therapeutic agents comprise:
a compound that increases the concentration of one or more of the anti-inflammatory/anti-proliferative PEDF protein;
a compound that increases the concentration of the anti-proliferative KLF4 protein;
a compound that increases the concentration of the anti-proliferative/anti-angiogenic/growth factor binder BTG2 protein;
a compound that increases the concentration of the anti-proliferative/angiogenesis inhibitor/growth factor binder Perlecan protein; and
combinations thereof; and
positioning the intravascular treatment device at a site of build-up of atherosclerotic plaque in a blood vessel, wherein the intravascular treatment device contacts the atherosclerotic site under conditions effective to transfer at least a portion of the one or more therapeutic agents to the subject.

2. The method of claim 1 wherein the atherosclerosis is associated with peripheral arterial disease.

3. The method of claim 1 or claim 2 wherein the one or more therapeutic agents are associated the intravascular treatment device such that when the device is positioned at a site of build-up of atherosclerotic plaque, the one or more therapeutic agents are in contact with the atherosclerotic plaque.

4. The method of any one of claims 1 through 3 wherein the intravascular treatment device comprises a polymeric coating comprising the one or more therapeutic agents.

5. The method of any one of claims 1 through 4 wherein the intravascular treatment device comprises a structural polymeric component comprising the one or more therapeutic agents.

6. The method of any one of claims 1 through 5 wherein the intravascular treatment device comprises a mixture of the one or more therapeutic agents.

7. The method of any one of claims 1 through 6 wherein the intravascular treatment device comprises a stent, a stent graft, an angioplasty balloon, or a combination thereof.

8. The method of claim 7 wherein the intravascular treatment device comprises a stent.

9. The method of claim 7 wherein the intravascular treatment device comprises an angioplasty balloon.

10. The method of any one of claims 1 through 9 wherein the one or more therapeutic agents comprise a combination of two or more therapeutic agents.

11. The method of claim 1 through 10 wherein the one or more therapeutic agents comprise:
a compound that increases the concentration of one or more of the anti-inflammatory/anti-proliferative PEDF protein;
a compound that increases the concentration of the anti-proliferative KLF4 protein; and
combinations thereof.

12. The method of claim 11 wherein the one or more therapeutic agents comprise PEDF protein (SEQ ID NO:1), PEDF 44 AA peptide (SEQ ID NO:2), an adenovial vector encoding PEDF (SEQ ID NO:1), LOR-253, and combinations thereof.

13. The method of any one of claims 1 through 12 wherein the intravascular treatment device further comprises a carrier for the one or more therapeutic agents.

14. The method of claim 13 wherein the carrier comprises an organic polymeric material.

15. The method of any one of claims 1 through 14 wherein the intravascular treatment device further comprises an excipient mixed with the one or more therapeutic agents.

16. An intravascular treatment device locatable at an atherosclerotic site in a blood vessel; wherein the device comprises one or more therapeutic agents comprising:
a compound that increases the concentration of one or more of the anti-inflammatory/anti-proliferative PEDF protein;
a compound that increases the concentration of the anti-proliferative KLF4 protein;
a compound that increases the concentration of the anti-proliferative/anti-angiogenic/growth factor binder BTG2 protein;
a compound that increases the concentration of the anti-proliferative/angiogenesis inhibitor/growth factor binder Perlecan protein; and
combinations thereof.

17. The device of claim 16 wherein the intravascular treatment device comprises a stent, a stent graft, an angioplasty balloon, and combinations thereof.

18. The device of claim 17 wherein the intravascular treatment device comprises a stent.

19. The device of claim 17 wherein the intravascular treatment device comprises an angioplasty balloon.

20. The device of any one of claims 16 through 19 wherein the one or more therapeutic agents are associated the intravascular treatment device such that when the device is positioned at a site of build-up of atherosclerotic plaque, the one or more therapeutic agents are in contact with the atherosclerotic plaque.

21. The device of any one of claims 16 through 19 wherein the one or more therapeutic agents comprise a combination of two or more therapeutic agents.

22. The device of any one of claims 16 through 21 wherein the one or more therapeutic agents comprise:
a compound that increases the concentration of one or more of the anti-inflammatory/anti-proliferative PEDF protein;
a compound that increases the concentration of the anti-proliferative KLF4 protein; and
combinations thereof.

23. The device of claim 22 wherein the one or more therapeutic agents comprise PEDF protein (SEQ ID NO:1), PEDF 44 AA peptide (SEQ ID NO:2), an adenovial vector encoding PEDF (SEQ ID NO:1), LOR-253, and combinations thereof.

24. The device of any one of claims 16 through 23 wherein the intravascular treatment device further comprises a carrier for the one or more therapeutic agents.

25. The device of claim 24 wherein the carrier comprises an organic polymeric material.

26. The device of any one of claims 16 through 23 wherein the intravascular treatment device comprises a polymeric coating comprising the one or more therapeutic agents.

27. The device of any one of claims 16 through 23 wherein the intravascular treatment device comprises a structural polymeric component comprising the one or more therapeutic agents.

28. The device of any one of claims 16 through 27 wherein the intravascular treatment device further comprises an excipient mixed with the one or more therapeutic agents.
